SEQUENCE LISTING (3 sequences)

SEQ ID NO: 1 - protein, 124 aa, Artificial Sequence
  Synthetic
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSS

SEQ ID NO: 2 - protein, 124 aa, Artificial Sequence
  Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 3 - protein, 108 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

(19) United States
(12) Patent Application Publication
(10) Pub. No.: US 2020/0399372 A1
(43) Pub. Date: Dec. 24, 2020

(54) USE OF BISPECIFIC ANTIGEN-BINDING MOLECULES THAT BIND PSMA AND CD3 IN COMBINATION WITH 4-1BB CO-STIMULATION

(71) Applicant: REGENERON PHARMACEUTICALS, INC., Tarrytown, NY (US)

(72) Inventors: Jessica R. Kirshner, New York, NY (US); Alison Crawford, Dobbs Ferry, NY (US); Danica Chiu, New York, NY (US)

(21) Appl. No.: 16/906,943

(22) Filed: Jun. 19, 2020

Related U.S. Application Data
(60) Provisional application No. 62/864,999, filed on Jun. 21, 2019.

(51) Int. Cl. C07K 16/28; C07K 16/30; A61K 47/68; A61K 51/10

(52) U.S. Cl. CPC C07K 16/2809; C07K 16/3069; C07K 16/2878; A61K 47/6887; C07K 2317/56; A61K 47/6849; A61K 51/1042; A61K 51/1072; C07K 2317/31; A61K 47/6869

(57) ABSTRACT

Provided herein are methods of treating cancer using bispecific antigen-binding molecules that bind to prostate-specific membrane antigen (PSMA) and CD3. According to certain embodiments, the antibodies useful herein bind human PSMA with high affinity and bind CD3 to induce human T cell proliferation. According to certain embodiments, bispecific antigen-binding molecules comprising a first antigen-binding domain that specifically binds human CD3, and a second antigen-binding molecule that specifically binds human PSMA are particularly useful herein. In certain embodiments, the bispecific antigen-binding molecules in combination with an anti-4-1BB agonist are capable of inhibiting the growth of prostate tumors expressing PSMA. The bispecific antigen-binding molecules in combination with an anti-4-1BB agonist are useful for the treatment of diseases and disorders in which an upregulated or induced targeted immune response is desired and/or therapeutically beneficial, for example, in the treatment of various cancers.

### CROSS-REFERENCE TO RELATED APPLICATIONS

This application claims the benefit under 35 U.S.C. § 119(e) of U.S. Provisional Application Ser. No. 62/864,999, filed Jun. 21, 2019, which is herein specifically incorporated by reference in its entirety.

### FIELD OF THE INVENTION

The present invention relates to bispecific antigen-binding molecules that bind prostate-specific membrane antigen (PSMA) and CD3 in combination with 4-1BB co-stimulation, and methods of use thereof.

### REFERENCE TO A SEQUENCE LISTING

An official copy of the sequence listing is submitted concurrently with the specification electronically via EFS-Web as an ASCII formatted sequence listing with a file name of 10595US01_SEQ_LIST_ST25, a creation date of Jun. 19, 2020, and a size of about 4,096 bytes. The sequence listing contained in this ASCII formatted document is part of the specification and is herein incorporated by reference in its entirety.

### BACKGROUND

Prostate-specific membrane antigen (PSMA), also known as folate hydrolase 1 (FOLH1), is an integral, non-shed membrane glycoprotein that is highly expressed in prostate epithelial cells and is a cell-surface marker for prostate cancer. Its expression is maintained in castrate-resistant prostate cancer, a condition with poor outcome and limited treatment options. Methods for treating prostate cancer by targeting PSMA have been investigated. For example, Yttrium-90 capromab is a radiotherapeutic comprising a monoclonal antibody to an intracellular epitope of PSMA; J591, a monoclonal antibody to an extracellular epitope of PSMA, is part of the radiotherapeutic Lutetium-177 J591; and MLN2704, in which maytansinoid 1 (DM1, an antimicrotubule agent) is conjugated to J591. These therapies have been associated with toxicity. PSMA is also expressed within the neovasculature of other tumors such as bladder, renal, gastric, and colorectal carcinomas.

CD3 is a homodimeric or heterodimeric antigen expressed on T cells in association with the T cell receptor complex (TCR) and is required for T cell activation. Functional CD3 is formed from the dimeric association of two of four different chains: epsilon, zeta, delta and gamma. The CD3 dimeric arrangements include gamma/epsilon, delta/epsilon, and zeta/zeta. Antibodies against CD3 have been shown to cluster CD3 on T cells, thereby causing T cell activation in a manner similar to the engagement of the TCR by peptide-loaded MHC molecules. Thus, anti-CD3 antibodies have been proposed for therapeutic purposes involving the activation of T cells. In addition, bispecific antibodies that are capable of binding CD3 and a target antigen have been proposed for therapeutic uses involving targeting T cell immune responses to tissues and cells expressing the target antigen.

In T-cell activation, co-stimulation via the TNF-receptor superfamily is key to survival, acquisition of effector functions, and memory differentiation. 4-1BB (Tnfrsf9), also known as CD137, is a member of the TNF-receptor superfamily. Receptor expression is induced by lymphocyte activation following TCR-mediated priming, but its levels can be augmented by CD28 co-stimulation. Exposure to ligand or agonist monoclonal antibodies (mAb) on CD8⁺ T cells costimulates 4-1BB, contributing to the clonal expansion, survival, and development of T cells, induced proliferation in peripheral monocytes, activation of NF-kappaB, enhanced T cell apoptosis induced by TCR/CD3 triggered activation, memory generation, and regulation of CD28 co-stimulation to promote Th1 cell responses.

### BRIEF SUMMARY

Provided herein are methods for treating a cancer in a subject. In some aspects, the methods comprise administering to the subject a pharmaceutical composition comprising an anti-PSMA/anti-CD3 bispecific antigen-binding molecule, or an anti-PSMA antibody, and a pharmaceutically acceptable carrier or diluent, and further administering to the subject an anti-4-1BB agonist. In some aspects, the methods comprise administering to the subject a pharmaceutical composition comprising an anti-PSMA/anti-CD3 bispecific antigen-binding molecule, or an anti-PSMA antibody, an anti-4-1BB agonist, and a pharmaceutically acceptable carrier or diluent. In some embodiments, the cancer is selected from the group consisting of prostate cancer, kidney cancer, bladder cancer, colorectal cancer, and gastric cancer. In some cases, the cancer is prostate cancer. In some cases, the prostate cancer is castrate-resistant prostate cancer.

Further provided herein are methods of treating a cancer or inhibiting the growth of a tumor. In some aspects, the methods comprise administering to a subject in need thereof a therapeutically effective amount of each of (a) an anti-PSMA antibody or antigen-binding fragment thereof or an anti-CD3/anti-PSMA bispecific antigen-binding molecule; and (b) an anti-4-1BB agonist.

Also provided herein are therapeutic methods for targeting/killing tumor cells expressing PSMA. In some aspects, the therapeutic methods comprise administering a therapeutically effective amount of an anti-CD3/anti-PSMA bispecific antigen-binding molecule, or an anti-PSMA antibody, and a therapeutically effective amount of an anti-4-1BB agonist to a subject in need thereof. In some aspects, the anti-CD3/anti-PSMA bispecific antigen-binding molecule, or the anti-PSMA antibody, and the anti-4-1BB agonist are formulated separately. In some aspects, the anti-CD3/anti-PSMA bispecific antigen-binding molecule, or the anti-PSMA antibody, and the anti-4-1BB agonist are formulated in the same pharmaceutical composition.

Also provided herein is the use of an anti-CD3/anti-PSMA bispecific antigen-binding molecule, or an anti-PSMA antibody, with an anti-4-1BB agonist in the manufacture of a medicament for the treatment of a disease or disorder related to or caused by PSMA-expressing cells.

Administration of an anti-PSMA antibody or antigen-binding fragment thereof, or an anti-PSMA/anti-CD3 bispecific antibody, to a subject in need thereof in combination with an anti-4-1BB agonist can decrease tumor volume relative to treatment in the absence of an anti-4-1BB agonist.

Administration of an anti-PSMA antibody or antigen-binding fragment thereof, or an anti-PSMA/anti-CD3 bispecific antibody, to a subject in need thereof in combination with an anti-4-1BB agonist can increase tumor free survival relative to treatment in the absence of an anti-4-1BB agonist.

Administration of an anti-PSMA antibody or antigen-binding fragment thereof, or an anti-PSMA/anti-CD3 bispecific antibody, to a subject in need thereof in combination with an anti-4-1BB agonist can increase TRAF1 expression in a tumor by at least about 4 fold relative to TRAF1 expression in the tumor of a subject administered the anti-CD3/anti-PSMA bispecific antigen-binding molecule in the absence of an anti-4-1BB agonist.

Administration of an anti-PSMA antibody or antigen-binding fragment thereof, or an anti-PSMA/anti-CD3 bispecific antibody, to a subject in need thereof in combination with an anti-4-1BB agonist can increase expression of Bcl2 in the tumor by at least about 2 fold relative to Bcl2 expression in the tumor of a subject administered the anti-CD3/anti-PSMA bispecific antigen-binding molecule in the absence of an anti-4-1BB agonist.

Administration of an anti-PSMA antibody or antigen-binding fragment thereof, or an anti-PSMA/anti-CD3 bispecific antibody, to a subject in need thereof in combination with an anti-4-1BB agonist can increase expression of BFL-1 in the tumor by at least about 3 fold relative to BFL-1 expression in the tumor of a subject administered the anti-CD3/anti-PSMA bispecific antigen-binding molecule in the absence of an anti-4-1BB agonist.

Administration of an anti-PSMA antibody or antigen-binding fragment thereof, or an anti-PSMA/anti-CD3 bispecific antibody, to a subject in need thereof in combination with an anti-4-1BB agonist can increase expansion of CD8+ T cells in the tumor and/or an increase in survival of CD8+ T cells relative to CD8+ T cells in the tumor of a subject administered the anti-CD3/anti-PSMA bispecific antigen-binding molecule in the absence of an anti-4-1BB agonist.

An anti-4-1BB agonist can be a small molecule or biologic agonist of 4-1BB, and in some aspects is an antibody. Exemplary anti-4-1BB agonists include commercially available antibodies, for example anti-mouse 4-1BB, and therapeutic antibodies such as urelumab and utomilumab.

Useful according to the methods provided herein are anti-PSMA antibodies or antigen-binding fragments thereof and bispecific antibodies and antigen-binding fragments thereof that bind human PSMA and human CD3. The bispecific antibodies are useful, inter alia, for targeting T cells expressing CD3, and for stimulating T cell activation, e.g., under circumstances where T cell-mediated killing of cells expressing PSMA is beneficial or desirable. For example, the bispecific antibodies can direct CD3-mediated T cell activation to specific PSMA-expressing cells, such as prostate tumor cells.

Anti-PSMA antibodies or antigen-binding fragments thereof that bind PSMA are useful in combination with an anti-4-1BB agonist for treating diseases and disorders related to or caused by PSMA-expressing tumors, and particularly, tumors that are larger and/or more difficult to treat. Exemplary anti-PSMA antibodies and antigen-binding fragments thereof are described in detail in U.S. Pat. No. 10,179,819. In some aspects, the anti-PSMA antibody comprises an HCVR of SEQ ID NO: 66 and a common light chain of SEQ ID NO: 1386 referred to in U.S. Pat. No. 10,179,819. In some aspects, the anti-PSMA antibody is the H1H11810P antibody referred to in U.S. Pat. No. 10,179,819.

Bispecific antigen-binding molecules (e.g., antibodies) that bind PSMA and CD3 are also referred to herein as “anti-PSMA/anti-CD3 bispecific molecules,” “anti-CD3/anti-PSMA bispecific molecules,” “PSMAxCD3 bsAbs”, or simply “PSMAxCD3”. The anti-PSMA portion of the anti-PSMA/anti-CD3 bispecific molecule is useful for targeting cells (e.g., tumor cells) that express PSMA (e.g., prostate tumors), and the anti-CD3 portion of the bispecific molecule is useful for activating T-cells. The simultaneous binding of PSMA on a tumor cell and CD3 on a T-cell facilitates directed killing (cell lysis) of the targeted tumor cell by the activated T-cell. The anti-PSMA/anti-CD3 bispecific molecules useful herein are therefore useful, inter alia, for treating diseases and disorders related to or caused by PSMA-expressing tumors (e.g., prostate cancers). The anti-PSMA/anti-CD3 bispecific molecules are also useful in combination with an anti-4-1BB agonist for treating diseases and disorders related to or caused by PSMA-expressing tumors, and particularly, tumors that are larger and/or more difficult to treat.

The bispecific antigen-binding molecules comprise a first antigen-binding domain that specifically binds human CD3, and a second antigen-binding domain that specifically binds PSMA.

Exemplary bispecific antibodies useful according to the methods provided herein are anti-CD3/anti-PSMA bispecific molecules, wherein the first antigen-binding domain that specifically binds CD3 comprises any of the HCVR amino acid sequences, any of the LCVR amino acid sequences, any of the HCVR/LCVR amino acid sequence pairs, any of the heavy chain CDR1-CDR2-CDR3 amino acid sequences, or any of the light chain CDR1-CDR2-CDR3 amino acid sequences as set forth in US publication 2014/0088295.

Useful according to the methods provided herein are anti-CD3/anti-PSMA bispecific antigen-binding molecules, wherein the first antigen-binding domain that specifically binds CD3 comprises any of the HCVR amino acid sequences and/or any of the LCVR amino acid sequences, or a substantially similar sequence thereof having at least 90%, at least 95%, at least 98% or at least 99% sequence identity, as set forth in Tables 12, 14, 15, 18, and 20 of U.S. Pat. No. 10,179,819. In some aspects, the first antigen-binding domain that specifically binds CD3 comprises a heavy chain variable region (HCVR-1) amino acid sequence of SEQ ID NO: 2.

Useful according to the methods provided herein are anti-CD3/anti-PSMA bispecific molecules, wherein the second antigen-binding domain that specifically binds PSMA comprises any of the HCVR amino acid sequences and/or any of the LCVR amino acid sequences, or a substantially similar sequence thereof having at least 90%, at least 95%, at least 98%, or at least 99% sequence identity, as set forth in Table 1 of U.S. Pat. No. 10,179,819. In some aspects, the second antigen-binding domain that specifically binds PSMA comprises a heavy chain variable region (HCVR-2) amino acid sequence of SEQ ID NO: 1.

Useful according to the methods provided herein are anti-CD3/anti-PSMA bispecific molecules, wherein the first antigen-binding domain that specifically binds CD3 comprises a HCVR-1 amino acid sequence of SEQ ID NO: 2 and wherein the second antigen-binding domain that specifically binds PSMA comprises a HCVR-2 amino acid sequence of SEQ ID NO: 1. In some aspects, the anti-CD3/anti-PSMA bispecific molecule comprises a common light chain variable region (LCVR) amino acid sequence of SEQ ID NO: 3.

In one aspect, provided herein is a pharmaceutical composition comprising an anti-PSMA antigen-binding molecule or anti-PSMA/anti-CD3 bispecific antigen-binding molecule and a pharmaceutically acceptable carrier or diluent. In some aspects, the pharmaceutical composition further comprises an anti-4-1BB agonist.

Useful according to the methods of the present disclosure are anti-PSMA antibodies and antigen-binding fragments thereof and anti-CD3/anti-PSMA bispecific antigen-binding molecules having a modified glycosylation pattern. In some applications, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In one aspect, the disclosure provides a pharmaceutical composition comprising an anti-PSMA antibody or antigen-binding fragment thereof or an anti-CD3/anti-PSMA bispecific antigen-binding molecule as disclosed herein, an anti-4-1BB agonist, and a pharmaceutically acceptable carrier. In a related aspect, the disclosure features a composition which is a combination of an anti-CD3/anti-PSMA bispecific antigen-binding molecule, an anti-4-1BB agonist, and a third therapeutic agent. In one embodiment, the third therapeutic agent is any agent that is advantageously combined with an anti-CD3/anti-PSMA bispecific antigen-binding molecule. Exemplary agents that may be advantageously combined with an anti-CD3/anti-PSMA bispecific antigen-binding molecule are discussed in detail elsewhere herein.

In another aspect, provided herein are radiolabeled anti-PSMA antibody conjugates and anti-CD3/anti-PSMA bispecific antigen-binding molecule conjugates for use in immuno-PET imaging. The conjugate comprises an anti-PSMA antibody or an anti-CD3/anti-PSMA bispecific antigen-binding molecule, a chelating moiety, and a positron emitter.

Provided herein are processes for synthesizing said conjugates and synthetic intermediates useful for the same.

Provided herein are methods of imaging a tissue that expresses PSMA, the methods comprising administering a radiolabeled anti-PSMA antibody conjugate or an anti-CD3/anti-PSMA bispecific antigen-binding molecule conjugate described herein to the tissue; and visualizing the PSMA expression by positron emission tomography (PET) imaging.

Provided herein are methods of imaging a tissue comprising PSMA-expressing cells, the methods comprising administering a radiolabeled anti-PSMA antibody conjugate or an anti-CD3/anti-PSMA bispecific antigen-binding molecule conjugate described herein to the tissue, and visualizing the PSMA expression by PET imaging.

Provided herein are methods for detecting PSMA in a tissue, the methods comprising administering a radiolabeled anti-PSMA antibody conjugate or an anti-CD3/anti-PSMA bispecific antigen-binding molecule conjugate described herein to the tissue; and visualizing the PSMA expression by PET imaging. In one embodiment, the tissue is present in a human subject. In certain embodiments, the subject is a non-human mammal. In certain embodiments, the subject has a disease or disorder such as cancer, an inflammatory disease, or an infection.

Provided herein are methods for detecting PSMA in a tissue, the methods comprising contacting the tissue with an anti-PSMA antibody or an anti-CD3/anti-PSMA bispecific antigen-binding molecule conjugated to a fluorescent molecule described herein; and visualizing the PSMA expression by fluorescence imaging.

Provided herein are methods for identifying a subject to be suitable for anti-tumor therapy, the methods comprising selecting a subject with a solid tumor, administering a radiolabeled anti-PSMA antibody conjugate or an anti-CD3/anti-PSMA bispecific antigen-binding molecule conjugate described herein, and visualizing the administered radiolabeled antibody conjugate in the tumor by PET imaging wherein presence of the radiolabeled antibody conjugate in the tumor identifies the subject as suitable for anti-tumor therapy.

Provided herein are methods of treating a tumor, the methods comprising selecting a subject with a solid tumor; determining that the solid tumor is PSMA positive; and administering an anti-tumor therapy to the subject in need thereof. In certain embodiments, the anti-tumor therapy comprises an inhibitor of the PD-1/PD-L1 signaling axis (e.g., an anti-PD-1 antibody or an anti-PD-L1 antibody), an example of a checkpoint inhibitor therapy. In certain embodiments, the subject is administered a radiolabeled anti-PSMA antibody conjugate or anti-CD3/anti-PSMA bispecific antigen-binding molecule conjugate described herein, and localization of the radiolabeled antibody conjugate is imaged via positron emission tomography (PET) imaging to determine if the tumor is PSMA positive. In certain embodiments, the subject is further administered a radiolabeled anti-PD-1 antibody conjugate, and localization of the radiolabeled antibody conjugate is imaged via positron emission tomography (PET) imaging to determine if the tumor is PD-1-positive.

Provided herein are methods for monitoring the efficacy of an anti-tumor therapy in a subject, wherein the methods comprise selecting a subject with a solid tumor wherein the subject is being treated with an anti-tumor therapy; administering a radiolabeled anti-PSMA antibody conjugate or an anti-CD3/anti-PSMA bispecific antigen-binding molecule conjugate described herein to the subject; imaging the localization of the administered radiolabeled conjugate in the tumor by PET imaging; and determining tumor growth, wherein a decrease from the baseline in uptake of the conjugate or radiolabeled signal indicates efficacy of the anti-tumor therapy.

In certain embodiments, the anti-tumor therapy comprises a PD-1 inhibitor (e.g., REGN2810, BGB-A317, nivolumab, pidilizumab, and pembrolizumab), a PD-L1 inhibitor (e.g., atezolizumab, avelumab, durvalumab, MDX-1105, and REGN3504, as well as those disclosed in Patent Publication No. US 2015-0203580), CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to LAGS, CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen-binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), a CD20 inhibitor (e.g., an anti-CD20 antibody such as rituximab), an antibody to a tumor-specific antigen [e.g., CA9, CA125, melanoma-associated antigen 3 (MAGES), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3×CD20 bispecific antibody, or PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, and an antibody-drug conjugate (ADC) (e.g., anti-CD19-DM4 ADC, and anti-DS6-DM4 ADC).

Provided herein are methods of increasing expansion of CD8+ T cells in tumor tissue. In some aspects, the methods comprise administering to a subject in need thereof a therapeutically effective amount of each of (a) an anti-CD3/anti-PSMA bispecific antigen-binding molecule; and (b) an anti-4-1BB agonist.

Provided herein are methods of eliciting and/or enhancing T cell responses to tumors. In some aspects, the methods comprise administering to a subject in need thereof a therapeutically effective amount of each of (a) an anti-CD3/anti-PSMA bispecific antigen-binding molecule; and (b) an anti-4-1BB agonist.

In some aspects, the CD8+ T cells to Treg ratio increases in the tumor tissue relative to the CD8+ T cells to Treg ratio in a tumor tissue in a subject administered an anti-CD3/anti-PSMA bispecific antigen-binding molecule in the absence of an anti-4-1BB agonist. In some aspects subsequent exposure to tumor cells elicits a memory response in the subject treated with the anti-CD3/anti-PSMA bispecific antigen-binding molecule in the presence of an anti-4-1BB agonist.

Other embodiments will become apparent from a review of the ensuing detailed description.

### BRIEF DESCRIPTION OF THE DRAWINGS

FIGS. 1A-1G show that PSMAxCD3 bispecific antibody can bind to both low and high antigen expressing cell lines and demonstrate that PSMAxCD3 bispecific antibody is able to induce target dependent, CD3-mediated T cell activation resulting in killing of PSMA expressing tumor cells. Data shown are from two wells combined and are representative of three independent experiments.

FIGS. 2A-2B demonstrate growth inhibition of human prostate cancer cells in a xenogeneic tumor model as a result of treatment with PSMAxCD3 bispecific antibody. In FIG. 2A, NSG mice were co-implanted with 22Rv1 cells and human PBMCs subcutaneously. Mice were dosed on days 0, 3 and 7 with 0.1, 1 mg/kg of PSMAxCD3 or 1 mg/kg of CD3-binding control. In FIG. 2B, NSG mice were co-implanted with C4-2 cells and human PBMCs subcutaneously. Mice were dosed on days 0, 3 and 7 with 0.01, 0.1 mg/kg of PSMAxCD3 or 0.1 mg/kg of CD3-binding control. Mean tumor volumes are shown as SEM (n=5, 3 replicates). ****P<0.0001. Statistical significance is measured by two-way ANOVA compared to CD3-binding control.

FIGS. 3A-3D show PSMA expression and accumulation of PSMAxCD3 bispecific antibody in PSMA expressing tissues of a HuT mouse and drug clearance. FIG. 3A shows relative PSMA expression in tissues of HuT mice by RT-PCR. FIGS. 3B and 3C show ex vivo tissue biodistribution measured on day 6 represented as percent injected dose per gram of tissue (% ID/g) and as tissue to blood ratio. Data shown as mean±SD. FIG. 3D shows PSMAxCD3 drug clearance over time measured in mice treated with 1 mg/kg of PSMAxCD3.

FIGS. 4A-4C demonstrate PSMAxCD3 bispecific antibody treatment effected prevention of tumor growth or growth delay in HuT mice implanted with a mouse prostate adenocarcinoma cell line expressing human PSMA in tumors that were smaller than 200 mm³. In larger tumors, a brief but transient anti-tumor response was observed. In FIG. 4A, mice were treated with 5 mg/kg of CD3-binding control (circle) or PSMAxCD3 (square) on days 0, 4, 7 and 11. 5/5 mice were tumor free. Mean tumor volumes are shown as SEM (n=7, 3 replicates). ****P<0.0001. In FIG. 4B, 50 mm³ tumors were treated with 5 mg/kg of CD3-binding control (circle) or PSMAxCD3 (square) on days 8, 12, 15, and 19. 2/5 mice were tumor free. Mean tumor volumes are shown as SEM (n=5, 3 replicates). ****P<0.0001. In FIG. 4C, 200 mm³ tumors were treated with 5 mg/kg of CD3-binding control (circle) or PSMAxCD3 (square) on days 9, 12, 16, and 19. 0/5 mice were tumor free. Mean tumor volumes are shown as SEM (n=5, 3 replicates). **P=0.0014.

FIGS. 5A-5D demonstrate the results of PSMAxCD3 bispecific antibody treatment of HuT mice having two different sized tumors on opposite flanks. The data show that the bispecific antibody targets tumors regardless of size but that efficacy is restricted to smaller tumors. FIG. 5A shows mean volume of small tumor is shown as SEM (n=5, three replicates). ***P<0.001. FIG. 5 B shows mean volume of large tumor shown as SEM (n=5, 3 replicates). *P=0.01. All statistical significance is measured by two-way ANOVA compared to CD3-binding control. FIGS. 5C and 5D show ex vivo tissue biodistribution measured on day 6 and represented as percent injected dose per gram of tissue (% ID/g) and as tissue to blood ratio after 1 mg/kg of ⁸⁹Zr-PSMAxCD3 or ⁸⁹Zr-CD3 binding control was administered to mice bearing small and large tumors. Data shown as mean±SD.

FIGS. 6A-6D demonstrate the anti-tumor efficacy of PSMAxCD3 bispecific antibody with anti-4-1BB co-stimulation in large TRAMP-C2hPMSA tumors (200 mm³). FIG. 6A shows representative flow plots and MFI of 4-1BB expression in tumor and splenic CD4 and CD8 T cells 48 hours after administration of 5 mg/kg of CD3-binding control or PSMAxCD3 (n=5). FIG. 6B shows established 200 mm³ TRAMP-C2-hPMSA tumors were treated once on day 9 with 5 mg/kg CD3-binding control (open circles), 2.5 mg/kg anti-4-1BB (solid circles), 1 mg/kg PSMAxCD3 (open triangles), 5 mg/kg PSMAxCD3 (closed triangles), 1 mg/kg PSMA+2.5 mg/kg of anti-4-1BB (open squares), or 5 mg/kg PSMAxCD3+2.5 mg/kg anti-4-1BB (closed squares). Mean tumor volume is shown as SEM (n=10, 3 replicates). ****P<0.0001. Statistical significance was measured by two-way ANOVA compared to CD3-binding control. FIG. 6C provides tumor free survival curves representing euthanasia of mice bearing tumors >2000 mm³. Significance is measured by Gehan-Breslow-Wilcoxon test compared to CD3-binding control. ****P<0.0001. Number of Tumor free (TF) mice are as follows: 0/10 for CD3 binding control; 0/10 for 5 mg/kg PSMAxCD3; 1/10 for 1 mg/kg PSMAxCD3; 2/10 for anti-4-1BB control; 6/10 for 1 mg/kg PSMA+2.5 mg/kg of anti-4-1BB; and 5/10 for 5 mg/kg PSMAxCD3+2.5 mg/kg anti-4-1BB. FIG. 6D provides relative expression of 4-1BB pathway genes in tumors 72 hours after treatment administration. (n=6) ****P<0.0001, ***P<0.009, *P<0.05. Statistical significance measured by one-way ANOVA.

FIGS. 7A-7B show increased CD8 T cells in tumor after combination therapy of PSMAxCD3 and anti-4-1BB as well as immunological memory. FIG. 7B mice that cleared the 50 mm³ tumor were re-challenged with TRAMP-C2-hPSMA tumor cells and were protected from a secondary tumor, indicating that tumor specific immunological memory can be induced with CD3-bispecific antibodies.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

As shown in the Examples, anti-tumor efficacy of PSMAxCD3 was observed against small tumors, though anti-tumor efficacy was greatly diminished against larger tumors, more realistically reflecting the challenges of treating solid tumors in the clinic.

As shown below, PSMAxCD3 bispecific antibodies resulted in CD8 T cell infiltration, activation and proliferation, which was efficacious in smaller tumors but not in larger tumors. The inventors sought to enhance and prolong PSMAxCD3 induced T cell activity by providing a costimulatory signal using an anti-4-1BB agonist. The 4-1BB signaling pathway can enhance the magnitude and duration of T cell responses by promoting T cell survival, reversing T cell anergy, and subsequently generating memory T cells to promote potent anti-tumor activity.

As shown herein, combining a PSMAxCD3 bispecific antibody with anti-4-1BB co-stimulation resulted in enhanced CD8 T cell infiltration, activation and proliferation resulting in striking anti-tumor efficacy in larger tumors with a single dose. This combination can also induce tumor-specific T cell memory.

The ability of anti-PSMA antibodies and PSMAxCD3 bispecific antibodies to activate intratumoral T cells and the ability of 4-1BB co-stimulation to enhance the magnitude and duration of the T cell response leading to remarkable anti-tumor efficacy are demonstrated herein. Combining anti-PSMA antibodies and PSMAxCD3-bispecific antibodies with 4-1BB co-stimulation is useful in methods of treating established solid tumors to achieve better overall survival.

Therapeutic Uses of the Antigen-Binding Molecules

The present disclosure includes methods comprising administering to a subject in need thereof an anti-PSMA antibody or antigen-binding fragment thereof, or a bispecific antigen-binding molecule that specifically binds CD3 and PSMA, with an anti-4-1BB agonist. A therapeutic composition useful according to the methods herein can comprise an anti-PSMA antibody or a PSMAxCD3-bispecific antigen-binding molecule and a pharmaceutically acceptable carrier or diluent. As used herein, the expression “a subject in need thereof” means a human or non-human animal that exhibits one or more symptoms or indicia of cancer (e.g., a subject expressing a tumor or suffering from any of the cancers mentioned herein below), or who otherwise would benefit from an inhibition or reduction in PSMA activity or a depletion of PSMA+ cells (e.g., prostate cancer cells).

The antibodies and bispecific antigen-binding molecules disclosed herein (and therapeutic compositions comprising the same) are useful, inter alia, in combination with an anti-4-1BB agonist for treating any disease or disorder in which stimulation, activation and/or targeting of an immune response would be beneficial. In particular, the anti-PSMA antibodies and anti-CD3/anti-PSMA bispecific antigen-binding molecules combined with the anti-4-1BB agonist may be used for the treatment, prevention and/or amelioration of any disease or disorder associated with or mediated by PSMA expression or activity or the proliferation of PSMA+ cells. The mechanism of action by which the therapeutic methods disclosed herein are achieved include killing of the cells expressing PSMA in the presence of effector cells, for example, by CDC, apoptosis, ADCC, phagocytosis, or by a combination of two or more of these mechanisms. Cells expressing PSMA which can be inhibited or killed using the antibodies or bispecific antigen-binding molecules include, for example, prostate tumor cells. Further therapeutic effect is achieved by 4-1BB co-stimulation, including contributing to the clonal expansion, survival, and development of T cells, induced proliferation in peripheral monocytes, activation of NF-kappaB, enhanced T cell apoptosis induced by TCR/CD3 triggered activation, and memory generation.

The antigen-binding molecules, including anti-PSMA antibodies and anti-PSMA/anti-CD3 bispecific antibodies, in combination with an anti-4-1BB agonist may be used to treat, e.g., primary and/or metastatic tumors arising in the gastrointestinal tract, prostate, kidney, and/or bladder. In certain embodiments, the antibodies or bispecific antigen-binding molecules are used to treat one or more of the following cancers: clear cell renal cell carcinoma, chromophobe renal cell carcinoma, (renal) oncocytoma, (renal) transitional cell carcinoma, prostate cancer, colorectal cancer, gastric cancer, urothelial carcinoma, (bladder) adenocarcinoma, or (bladder) small cell carcinoma. According to certain embodiments of the present disclosure, the anti-PSMA antibodies and anti-PSMA/anti-CD3 bispecific antibodies in combination with an anti-4-1BB agonist are useful for treating a patient afflicted with a castrate-resistant prostate cancer. According to other related embodiments disclosed herein, methods are provided comprising administering an anti-CD3/anti-PSMA bispecific antigen-binding molecule in combination with an anti-4-1BB agonist to a patient who is afflicted with a castrate-resistant prostate cancer.

The present disclosure also includes methods for treating established tumors in a subject, with established being defined as a measurable tumor, i.e., measurable in a way that's appropriate for a given cancer.

The present disclosure also includes methods for treating residual cancer in a subject. As used herein, the term “residual cancer” means the existence or persistence of one or more cancerous cells in a subject following treatment with an anti-cancer therapy.

According to certain aspects, the present disclosure provides methods for treating a disease or disorder associated with PSMA expression (e.g., prostate cancer) comprising administering one or more of the bispecific antigen-binding molecules described elsewhere in combination with an anti-4-1BB agonist to a subject after the subject has been determined to have prostate cancer (e.g., castrate-resistant prostate cancer). For example, the present disclosure includes methods for treating prostate cancer comprising administering an anti-CD3/anti-PSMA bispecific antigen-binding molecule to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received hormone therapy (e.g., anti-androgen therapy).

### Definitions

The expression “CD3,” as used herein, refers to an antigen which is expressed on T cells as part of the multimolecular T cell receptor (TCR) and which consists of a homodimer or heterodimer formed from the association of two of four receptor chains: CD3-epsilon, CD3-delta, CD3-zeta, and CD3-gamma. All references to proteins, polypeptides and protein fragments herein are intended to refer to the human version of the respective protein, polypeptide or protein fragment unless explicitly specified as being from a non-human species. Thus, the expression “CD3” means human CD3 unless specified as being from a non-human species, e.g., “mouse CD3,” “monkey CD3,” etc.

As used herein, “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). The antibodies and antigen-binding fragments useful herein may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the expression “cell surface-expressed CD3” means one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. “Cell surface-expressed CD3” includes CD3 proteins contained within the context of a functional T cell receptor in the membrane of a cell. The expression “cell surface-expressed CD3” includes CD3 protein expressed as part of a homodimer or heterodimer on the surface of a cell (e.g., gamma/epsilon, delta/epsilon, and zeta/zeta CD3 dimers). The expression, “cell surface-expressed CD3” also includes a CD3 chain (e.g., CD3-epsilon, CD3-delta or CD3-gamma) that is expressed by itself, without other CD3 chain types, on the surface of a cell. A “cell surface-expressed CD3” can comprise or consist of a CD3 protein expressed on the surface of a cell which normally expresses CD3 protein. Alternatively, “cell surface-expressed CD3” can comprise or consist of CD3 protein expressed on the surface of a cell that normally does not express human CD3 on its surface but has been artificially engineered to express CD3 on its surface.

The expression “PSMA,” as used herein, refers to prostate-specific membrane antigen, also known as folate hydrolase 1 (FOLH1). PSMA is an integral, non-shed membrane glycoprotein that is highly expressed in prostate epithelial cells and is a cell-surface marker for prostate cancer. PSMA is an attractive cell surface target for late-stage malignancies. It is also expressed on the neovasculature of clear cell renal carcinomas, bladder, colon and breast cancers.

The expression “4-1BB” as used herein, also known as CD137, refers to an activation-induced costimulatory molecule. 4-1BB is an important regulator of immune responses and is a member of the TNF-receptor superfamily. The expression “anti-4-1BB agonist” is any ligand that binds 4-1BB and activates the receptor. Exemplary anti-4-1BB agonists include urelumab (BMS-663513), and utomilumab (PF-05082566), and commercially available anti-mouse 4-1BB antibodies. In addition, the term “4-1BB agonist” refers to any molecule that partially or fully promotes, induces, increases, and/or activates a biological activity of 4-1BB. Suitable agonist molecules specifically include agonist antibodies or antibody fragments, including bispecific antibodies, e.g. a bispecific antibody comprising one arm that binds 4-1BB on an immune cell and the other arm binds to, for example, an antigen on a tumor target. The term also includes fragments or amino acid sequence variants of native polypeptides, peptides, antisense oligonucleotides, small organic molecules, etc. In some embodiments, activation in the presence of the agonist is observed in a dose-dependent manner. In some embodiments, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% higher than the signal measured with a negative control under comparable conditions. Efficacy of an agonist can also be determined using functional assays, such as the ability of an agonist to activate or promote the function of the polypeptide. For example, a functional assay may comprise contacting a polypeptide with a candidate agonist molecule and measuring a detectable change in one or more biological activities normally associated with the polypeptide. The potency of an agonist is usually defined by its EC₅₀ value (concentration required to activate 50% of the agonist response). The lower the EC₅₀ value the greater the potency of the agonist and the lower the concentration that is required to activate the maximum biological response. A 4-1BB agonist may also include a molecule containing the 4-1BB-Ligand or a fragment of the 4-1BB-Ligand, e.g., a bispecific molecule comprising one arm that contains 4-1BBL or fragment thereof and the other arm binds to, for example, an antigen on a tumor. These fragments may include an Fc region.

The term “antigen-binding molecule” includes antibodies and antigen-binding fragments of antibodies, including, e.g., bispecific antibodies.

The term “antibody”, as used herein, means any antigen-binding molecule or molecular complex comprising at least one complementarity determining region (CDR) that specifically binds to or interacts with a particular antigen (e.g., PSMA or CD3). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments disclosed herein, the FRs of the anti-PSMA antibody or anti-CD3 antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody useful herein include: (i) V_{H}-C_{H}1; V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody useful herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody useful herein using routine techniques available in the art.

The antibodies useful herein may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody disclosed herein in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments, anti-PSMA/anti-CD3 bispecific antibodies useful herein are human antibodies. The term “human antibody”, as used herein, is intended to include antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies useful according to the methods disclosed herein may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2 or C_{H}3 region which may be desirable, for example, in production, to improve the yield of the desired antibody form.

The antibodies useful herein may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-PSMA antibodies and anti-PSMA/anti-CD3 bispecific antibodies useful according to the methods disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies useful herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

Useful according to the methods provided herein are anti-PSMA/anti-CD3 antibodies comprising variants of any of the HCVR, LCVR, and/or CDR amino acid sequences disclosed herein having one or more conservative substitutions. For example, the present disclosure includes anti-PSMA/anti-CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR or LCVR amino acid sequences set forth in Table 1 herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence disclosed herein to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

Sequence Variants

The bispecific antibodies useful herein comprise one or more amino acid substitutions, insertions and/or deletions in the framework and/or CDR regions of the heavy chain variable domains as compared to the corresponding germline sequences from which the antibodies were derived.

Also useful herein are antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), and having weak or no detectable antigen binding.

Furthermore, the antibodies useful herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be tested for one or more desired properties such as, improved binding specificity, weak or reduced binding affinity, improved or enhanced pharmacokinetic properties, reduced immunogenicity, etc. Antibodies and antigen-binding fragments obtained in this general manner given the guidance of the present disclosure are encompassed within the present invention.

Useful according to the present disclosure are bispecific antibodies comprising variants of any of the HCVR or LCVR amino acid sequences provided herein having one or more conservative substitutions. The antibodies and bispecific antigen-binding molecules useful herein comprise one or more amino acid substitutions, insertions and/or deletions in the framework and/or CDR regions of the HCVR and LCVR as compared to the corresponding germline sequences from which the individual antigen-binding domains were derived, while maintaining or improving the desired antigen-binding characteristics. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

The present disclosure also includes antigen-binding molecules comprising an antigen-binding domain with an HCVR and/or CDR amino acid sequence that is substantially identical to any of the HCVR and/or CDR amino acid sequences disclosed herein, while maintaining or improving the desired antigen affinity. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence disclosed herein to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

Once obtained, antigen-binding domains that contain one or more germline mutations were tested for decreased binding affinity utilizing one or more in vitro assays. Although antibodies that recognize a particular antigen are typically screened for their purpose by testing for high (i.e. strong) binding affinity to the antigen, the antibodies useful herein exhibit weak binding or no detectable binding. Bispecific antigen-binding molecules comprising one or more antigen-binding domains obtained in this general manner are also encompassed within the present disclosure and were found to be advantageous as avidity-driven tumor therapies.

Unexpected benefits, for example, improved pharmacokinetic properties and low toxicity to the patient may be realized from the methods described herein.

Binding Properties of the Antibodies

As used herein, the term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, C A, J Immunol Methods. 1997, 201(2):223-31; Geuijen, C A, et al. J Immunol Methods. 2005, 302(1-2):68-77).

Accordingly, the antibody or antigen-binding protein disclosed herein binds to the predetermined antigen or cell surface molecule (receptor) having an affinity corresponding to a K_{D} value that is at least ten-fold lower than its affinity for binding to a non-specific antigen (e.g., BSA, casein). According to the present disclosure, the affinity of an antibody corresponding to a K_{D} value that is equal to or less than ten-fold lower than a non-specific antigen may be considered non-detectable binding, however such an antibody may be paired with a second antigen binding arm for the production of a bispecific antibody disclosed herein.

The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec −1 or 1/s) refers to the dissociation rate constant of a particular antibody-antigen interaction, or the dissociation rate constant of an antibody or antibody-binding fragment. Said value is also referred to as the k_{off} value.

The term “kₐ” (M−1×sec−1 or 1/M) refers to the association rate constant of a particular antibody-antigen interaction, or the association rate constant of an antibody or antibody-binding fragment.

The term “K_{A}” (M−1 or 1/M) refers to the association equilibrium constant of a particular antibody-antigen interaction, or the association equilibrium constant of an antibody or antibody-binding fragment. The association equilibrium constant is obtained by dividing the kₐ by the k_{d}.

The term “EC50” or “EC₅₀” refers to the half maximal effective concentration, which includes the concentration of an antibody which induces a response halfway between the baseline and maximum after a specified exposure time. The EC₅₀ essentially represents the concentration of an antibody where 50% of its maximal effect is observed. In certain embodiments, the EC₅₀ value equals the concentration of an antibody disclosed herein that gives half-maximal binding to cells expressing CD3 or tumor-associated antigen, as determined by e.g. a FACS binding assay. Thus, reduced or weaker binding is observed with an increased EC₅₀, or half maximal effective concentration value.

In one embodiment, decreased binding can be defined as an increased EC₅₀ antibody concentration which enables binding to the half-maximal amount of target cells.

In another embodiment, the EC₅₀ value represents the concentration of an antibody that elicits half-maximal depletion of target cells by T cell cytotoxic activity. Thus, increased cytotoxic activity (e.g. T cell-mediated tumor cell killing) is observed with a decreased EC₅₀, or half maximal effective concentration value.

Bispecific Antigen-Binding Molecules

The antibodies useful herein may be monospecific, bi-specific, or multispecific.

Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-PSMA/anti-CD3 bispecific antibodies useful herein can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second or additional binding specificity.

Use of the expression “anti-CD3 antibody” or “anti-PSMA antibody” herein is intended to include both monospecific anti-CD3 or anti-PSMA antibodies as well as bispecific antibodies comprising a CD3-binding arm and a PSMA-binding arm. Thus, the present disclosure includes monospecific antibodies which bind PSMA, for example, those anti-PSMA antibodies described in U.S. Pat. No. 10,179,819. Exemplary anti-PSMA antibodies include the H1H11810P antibody and antibodies comprising the CDRs within the H1H11810 antibody as disclosed in U.S. Pat. No. 10,179,819. In addition, the present disclosure includes bispecific antibodies wherein one arm of an immunoglobulin binds human CD3, and the other arm of the immunoglobulin is specific for human PSMA. Exemplary sequences of the bispecific antibody useful according to the methods provided herein are shown in Table 1.

In certain embodiments, the CD3-binding arm binds to human CD3 and induces human T cell activation. In certain embodiments, the CD3-binding arm binds weakly to human CD3 and induces human T cell activation. In other embodiments, the CD3-binding arm binds weakly to human CD3 and induces tumor-associated antigen-expressing cell killing in the context of a bispecific or multispecific antibody. In other embodiments, the CD3-binding arm binds or associated weakly with human and cynomolgus (monkey) CD3, yet the binding interaction is not detectable by in vitro assays known in the art.

According to certain exemplary embodiments, the present disclosure includes bispecific antigen-binding molecules that specifically bind CD3 and PSMA. Such molecules may be referred to herein as, e.g., “anti-CD3/anti-PSMA,” or “anti-CD3×PSMA” or “CD3×PSMA” bispecific molecules, or other similar terminology (e.g., anti-PSMA/anti-CD3).

The term “PSMA,” as used herein, refers to the human PSMA protein unless specified as being from a non-human species (e.g., “mouse PSMA,” “monkey PSMA,” etc.).

The aforementioned bispecific antigen-binding molecules that specifically bind CD3 and PSMA may comprise an anti-CD3 antigen-binding molecule which binds to CD3 with a weak binding affinity such as exhibiting a K_{D} of greater than about 40 nM, as measured by an in vitro affinity binding assay.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bispecific antigen-binding molecule” means a protein, polypeptide or molecular complex comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. In the context of the present disclosure, the first antigen-binding domain specifically binds a first antigen (e.g., CD3), and the second antigen-binding domain specifically binds a second, distinct antigen (e.g., PSMA).

In certain exemplary embodiments, the bispecific antigen-binding molecule is a bispecific antibody. Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3.

The first antigen-binding domain and the second antigen-binding domain may be directly or indirectly connected to one another to form a bispecific antigen-binding molecule useful herein. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules useful herein will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules useful herein. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab^{e} bispecific formats (see, e.g., Klein et al. 2012, mAbs 4:6, 1-11, and references cited therein, for a review of the foregoing formats).

In the context of bispecific antigen-binding molecules useful herein, the multimerizing domains, e.g., Fc domains, may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., UR/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T2500 and M428L); and a 307 and/or 308 modification (e.g., 308F or 308P).

The present disclosure also includes bispecific antigen-binding molecules comprising a first Ig C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contain a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E4190, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]−[IgG4 upper hinge]−[IgG2 lower hinge]−[IgG4 C_{H}2]− [IgG4 C_{H}3]. Another example of a chimeric Fc domain that can be included in any of the antigen-binding molecules set forth herein comprises, from N- to C-terminus: [IgG1 C_{H}1]− [IgG1 upper hinge]−[IgG2 lower hinge]−[IgG4 C_{H}2]− [IgG1 C_{H}3]. These and other examples of chimeric Fc domains that can be included in any of the antigen-binding molecules useful herein are described in US Publication 2014/0243504, published Aug. 28, 2014, which is herein incorporated in its entirety. Chimeric Fc domains having these general structural arrangements, and variants thereof, can have altered Fc receptor binding, which in turn affects Fc effector function.

pH-Dependent Binding

The present disclosure includes anti-PSMA antibodies and anti-CD3/anti-PSMA bispecific antigen-binding molecules, with pH-dependent binding characteristics. For example, an anti-PSMA arm of a bispecific antigen-binding molecule useful herein may exhibit reduced binding to PSMA at acidic pH as compared to neutral pH. Alternatively, anti-CD3/anti-PSMA bispecific antigen-binding molecules useful herein may exhibit enhanced binding to PSMA at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to PSMA at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

Antibodies Comprising Fc Variants

According to certain embodiments useful herein, anti-PSMA antibodies and anti-CD3/anti-PSMA bispecific antigen-binding molecules are provided comprising an Fc domain comprising one or more mutations which enhance or diminish antibody binding to the FcRn receptor, e.g., at acidic pH as compared to neutral pH. For example, the present disclosure includes antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present disclosure includes anti-PSMA antibodies and anti-CD3/anti-PSMA bispecific antigen-binding molecules, comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present disclosure.

Biological Characteristics of the Antibodies and Bispecific Antigen-Binding Molecules

Useful according to the present disclosure are monospecific and bispecific antibodies and antigen-binding fragments thereof that bind CD3-expressing human T-cells and/or human PSMA with high affinity (e.g., sub-nanomolar K_{D} values). Such antibodies and their properties are disclosed in U.S. Pat. No. 10,179,819, incorporated by reference herein. Such bispecific antibodies are particularly useful in combination with an anti-4-1BB agonist in the treatment of tumors.

Useful herein are anti-PSMA antibodies and anti-CD3/anti-PSMA bispecific antigen-binding molecules which exhibit one or more characteristics selected from the group consisting of: (a) inhibiting tumor growth in immunocompromised mice bearing human prostate cancer xenografts; (b) inhibiting tumor growth in immunocompetent mice bearing human prostate cancer xenografts; (c) suppressing tumor growth in immunocompromised mice bearing human prostate cancer xenografts; and (d) reducing tumor growth of established tumors in immunocompetent mice bearing human prostate cancer xenografts (see, e.g., U.S. Pat. No. 10,179,819, Example 8).

Useful herein are antibodies and antigen-binding fragments thereof that bind human CD3 with medium or low affinity, depending on the therapeutic context and particular targeting properties that are desired. For example, in the context of a bispecific antigen-binding molecule, wherein one arm binds CD3 and another arm binds a target antigen (e.g., PSMA), it may be desirable for the target antigen-binding arm to bind the target antigen with high affinity while the anti-CD3 arm binds CD3 with only moderate or low affinity. In this manner, preferential targeting of the antigen-binding molecule to cells expressing the target antigen may be achieved while avoiding general/untargeted CD3 binding and the consequent adverse side effects associated therewith.

The bispecific antigen-binding molecules (e.g., bispecific antibodies) useful herein are capable of simultaneously binding to human CD3 and a human PSMA. The binding arm that interacts with cells that express CD3 may have weak to no detectable binding as measured in a suitable in vitro binding assay. The extent to which a bispecific antigen-binding molecule binds cells that express CD3 and/or PSMA can be assessed by fluorescence activated cell sorting (FACS), as illustrated in U.S. Pat. No. 10,179,819, Example 5.

For example, useful herein are bispecific antibodies thereof which specifically bind human T-cell lines which express CD3 but do not express PSMA (e.g., Jurkat), primate T-cells (e.g., cynomolgus peripheral blood mononuclear cells [PBMCs]), and/or PSMA-expressing cells. Useful herein are bispecific antigen-binding molecules which bind any of the aforementioned T cells and T cell lines with an EC₅₀ value of from about 1.8×10⁻⁸ (18 nM) to about 2.1×10⁻⁷ (210 nM), or more (i.e. weaker affinity), or EC₅₀ is undetectable, as determined using a FACS binding assay as set forth in U.S. Pat. No. 10,179,819, Example 5, or a substantially similar assay. Also useful herein are bispecific antibodies which bind to PSMA-expressing cells and cell lines, with an EC₅₀ value of less than or equal to 5.6 nM (5.6×10⁻⁹), as determined using a FACS binding assay as set forth in U.S. Pat. No. 10,179,819, Example 5, or a substantially similar assay.

In some aspects, the bispecific antibodies bind human CD3 with weak (i.e. low) or even no detectable affinity. According to certain embodiments, the present disclosure includes antibodies and antigen-binding fragments of antibodies that bind human CD3 (e.g., at 37° C.) with a K_{D} of greater than about 11 nM as measured by surface plasmon resonance.

In some aspects, the bispecific antibodies bind monkey (i.e. cynomolgus) CD3 with weak (i.e. low) or even no detectable affinity.

In some aspects, the bispecific antibodies bind human CD3 and induce T cell activation. For example, certain anti-CD3 antibodies induce human T cell activation with an EC₅₀ value of less than about 113 pM, as measured by an in vitro T cell activation assay.

The bispecific antibodies useful herein can bind human CD3 and induce T cell-mediated killing of tumor antigen-expressing cells. For example, the present disclosure includes bispecific antibodies that induce T cell-mediated killing of tumor cells with an EC₅₀ of less than about 1.3 nM, as measured in an in vitro T cell-mediated tumor cell killing assay.

The bispecific antibodies useful herein can bind CD3 with a dissociative half-life (t½) of less than about 10 minutes as measured by surface plasmon resonance at 25° C. or 37° C.

The anti-CD3/anti-PSMA bispecific antigen-binding molecules useful herein may additionally exhibit one or more characteristics selected from the group consisting of: (a) inducing PBMC proliferation in vitro; (b) activating T-cells via inducing IFN-gamma release and CD25 upregulation in human whole blood; and (c) inducing T-cell mediated cytotoxicity on anti-PSMA-resistant cell lines.

The present disclosure includes anti-CD3/anti-PSMA bispecific antigen-binding molecules which are capable of depleting tumor antigen-expressing cells in a subject (see, e.g., U.S. Pat. No. 10,179,819, Example 8). For example, according to certain embodiments, anti-CD3/anti-PSMA bispecific antigen-binding molecules are provided, wherein a single administration of 1 μg, or 10 μg, or 100 μg, or 1 mg, 3 mg, 5 mg, 10 mg, 30 mg, 50 mg, 100 mg, 300 mg, or 500 mg per patient of the bispecific antigen-binding molecule to a subject (e.g., at a dose of about 5 mg/kg, about 2.5 mg/kg, about 1 mg/kg, about 0.1 mg/kg, about 0.08 mg/kg, about 0.06 mg/kg, about 0.04 mg/kg, about 0.02 mg/kg, about 0.01 mg/kg, or less) causes a reduction in the number of PSMA-expressing cells in the subject (e.g., tumor growth in the subject is suppressed or inhibited) below detectable levels. In certain embodiments, a single administration of the anti-CD3/anti-PSMA bispecific antigen-binding molecule at a dose of about 0.4 mg/kg causes a reduction in tumor growth in the subject below detectable levels by about day 7, about day 6, about day 5, about day 4, about day 3, about day 2, or about day 1 after administration of the bispecific antigen-binding molecule to the subject. According to certain embodiments, a single administration of an anti-CD3/anti-PSMA bispecific antigen-binding molecule disclosed herein, at a dose of at least about 0.01 mg/kg, causes the number of PSMA-expressing tumor cells to remain below detectable levels until at least about 7 days, 8 days, 9 days, 10 days, 11 days, 12 days, 13 days, 14 days, 15 days, 16 days, 17 days or more, following the administration. As used herein, the expression “below detectable levels” means that no tumor cells can be directly or indirectly detected growing subcutaneously in a subject using standard caliper measurement methods, e.g., as set forth in U.S. Pat. No. 10,179,819 Example 8, herein.

Also useful according to the methods provided herein are anti-CD3/anti-PSMA bispecific antigen-binding molecules which exhibit one or more characteristics selected from the group consisting of: (a) inhibiting tumor growth in immunocompromised mice bearing human prostate cancer xenografts; (b) inhibiting tumor growth in immunocompetent mice bearing human prostate cancer xenografts; (c) suppressing tumor growth of tumors in immunocompromised mice bearing human prostate cancer xenografts; and (d) reducing tumor growth of established tumors in immunocompetent mice bearing human prostate cancer xenografts (see, e.g., U.S. Pat. No. 10,179,819, Example 8). Exemplary anti-CD3/anti-PSMA bispecific antigen-binding molecules can further exhibit one or more characteristics selected from the group consisting of: (a) induce transient dose-dependent increases in circulating cytokines, and (b) induce transient decreases in circulating T cells.

Epitope Mapping and Related Technologies

The epitope on CD3 and/or PSMA to which the antigen-binding molecules useful herein bind may consist of a single contiguous sequence of 3 or more (e.g., 3, 4, 5, 6, 7, 8, 9, 10, 11, 12, 13, 14, 15, 16, 17, 18, 19, 20 or more) amino acids of a CD3 or PSMA protein. Alternatively, the epitope may consist of a plurality of non-contiguous amino acids (or amino acid sequences) of CD3 or PSMA. The antibodies useful according to the methods disclosed herein may interact with amino acids contained within a single CD3 chain (e.g., CD3-epsilon, CD3-delta or CD3-gamma), or may interact with amino acids on two or more different CD3 chains. The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., N.Y.), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain of an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystallography of the antigen/antibody complex may also be used for epitope mapping purposes.

Exemplary bispecific antigen-binding molecules useful herein can comprise a first antigen-binding domain that specifically binds human CD3 and/or cynomolgus CD3 with low or detectable binding affinity, and a second antigen binding domain that specifically binds human PSMA, wherein the first antigen-binding domain binds to the same epitope on CD3 as any of the specific exemplary CD3-specific antigen-binding domains described herein, and/or wherein the second antigen-binding domain binds to the same epitope on PSMA as any of the specific exemplary PSMA-specific antigen-binding domains described herein.

Likewise, the bispecific antigen-binding molecules useful herein can comprise a first antigen-binding domain that specifically binds human CD3, and a second antigen binding domain that specifically binds human PSMA, wherein the first antigen-binding domain competes for binding to CD3 with any of the specific exemplary CD3-specific antigen-binding domains described herein in Table 1, and/or wherein the second antigen-binding domain competes for binding to PSMA with any of the specific exemplary PSMA-specific antigen-binding domains described herein in Table 1.

One can easily determine whether a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on PSMA (or CD3) as a reference bispecific antigen-binding molecule of the present disclosure, the reference bispecific molecule is first allowed to bind to a PSMA protein (or CD3 protein). Next, the ability of a test antibody to bind to the PSMA (or CD3) molecule is assessed. If the test antibody is able to bind to PSMA (or CD3) following saturation binding with the reference bispecific antigen-binding molecule, it can be concluded that the test antibody binds to a different epitope of PSMA (or CD3) than the reference bispecific antigen-binding molecule. On the other hand, if the test antibody is not able to bind to the PSMA (or CD3) molecule following saturation binding with the reference bispecific antigen-binding molecule, then the test antibody may bind to the same epitope of PSMA (or CD3) as the epitope bound by the reference bispecific antigen-binding molecule. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antigen-binding proteins bind to the same (or overlapping) epitope if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a PSMA protein (or CD3 protein) under saturating conditions followed by assessment of binding of the test antibody to the PSMA (or CD3) molecule. In a second orientation, the test antibody is allowed to bind to a PSMA (or CD3) molecule under saturating conditions followed by assessment of binding of the reference antigen-binding molecule to the PSMA (or CD3) molecule. If, in both orientations, only the first (saturating) antigen-binding molecule is capable of binding to the PSMA (or CD3) molecule, then it is concluded that the test antibody and the reference antigen-binding molecule compete for binding to PSMA (or CD3). As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

Preparation of Antigen-Binding Domains and Construction of Bispecific Molecules

Antigen-binding domains specific for particular antigens can be prepared by any antibody generating technology known in the art. Once obtained, two different antigen-binding domains, specific for two different antigens (e.g., CD3 and PSMA), can be appropriately arranged relative to one another to produce a bispecific antigen-binding molecule using routine methods. (A discussion of exemplary bispecific antibody formats that can be used to construct bispecific antigen-binding molecules of the present disclosure is provided elsewhere herein). In certain embodiments, one or more of the individual components (e.g., heavy and light chains) of the multispecific antigen-binding molecules are derived from chimeric, humanized or fully human antibodies. Methods for making such antibodies are well known in the art. For example, one or more of the heavy and/or light chains of the bispecific antigen-binding molecules useful herein can be prepared using VELOCIMMUNE™ technology. Using VELOCIMMUNE™ technology (see, for example, U.S. Pat. No. 6,596,541, Regeneron Pharmaceuticals, VELOCIMMUNE®, or any other human antibody generating technology), high affinity chimeric antibodies to a particular antigen (e.g., CD3 or PSMA) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules useful herein.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., U.S. Pat. No. 10,143,186 for a detailed discussion of such engineered mice and the use thereof to produce bispecific antigen-binding molecules).

Bioequivalents

The presently disclosed methods contemplate the use of antigen-binding molecules having amino acid sequences that vary from those of the exemplary molecules disclosed herein but that retain the ability to bind CD3 and/or PSMA. Such variant molecules may comprise one or more additions, deletions, or substitutions of amino acids when compared to parent sequence, but exhibit biological activity that is essentially equivalent to that of the described bispecific antigen-binding molecules.

Useful herein are antigen-binding molecules that are bioequivalent to any of the exemplary antigen-binding molecules set forth in Table 1. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

Species Selectivity and Species Cross-Reactivity

According to certain embodiments, bispecific antigen-binding molecules useful herein bind to human CD3 but not to CD3 from other species. Also useful herein are antigen-binding molecules which bind to human PSMA but not to PSMA from other species. The presently disclosed methods also contemplate use of bispecific antigen-binding molecules that bind to human CD3 and to CD3 from one or more non-human species; and/or bispecific antigen-binding molecules that bind to human PSMA and to PSMA from one or more non-human species.

According to certain exemplary embodiments, antigen-binding molecules useful herein bind to human CD3 and/or human PSMA and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CD3 and/or PSMA. For example, in a particular exemplary embodiment of the present disclosure bispecific antigen-binding molecules are provided comprising a first antigen-binding domain that binds human CD3 and cynomolgus CD3, and a second antigen-binding domain that specifically binds human PSMA.

Radiolabeled Immunoconjugates of Anti-PSMA/anti-CD3 Antigen Binding Molecule for Immuno-PET Imaging

Provided herein are radiolabeled antigen-binding proteins that bind an anti-PSMA antibody or an anti-PSMA/anti-CD3 antigen binding molecule. In some embodiments, the radiolabeled antigen-binding proteins comprise an antigen-binding protein covalently linked to a positron emitter. In some embodiments, the radiolabeled antigen-binding proteins comprise an antigen-binding protein covalently linked to one or more chelating moieties, which are chemical moieties that are capable of chelating a positron emitter.

Suitable radiolabeled antigen-binding proteins, e.g., radiolabeled antibodies, include those that do not impair, or do not substantially impair T-cell function upon exposure to the radiolabeled antigen-binding protein. In some embodiments, a radiolabeled antigen-binding protein that binds an anti-PSMA/anti-CD3 antigen binding molecule is a weak blocker of CD3 T-cell function, i.e. T-cell function is unimpaired, or substantially unimpaired, upon exposure to the radiolabeled antibody. Use of a radiolabeled anti-CD3 binding protein having minimal impact on CD3 mediated T-cell function according to methods provided herein ensures a subject treated with the molecule is not disadvantaged by the inability of its T-cells to clear infection.

In some embodiments, an anti-PSMA antibody or an anti-PSMA/anti-CD3 antigen binding molecule, e.g., bispecific antibodies, are provided, wherein said antigen-binding proteins are covalently bonded to one or more moieties having the following structure:

-L-M_{z}

wherein L is a chelating moiety; M is a positron emitter; and z, independently at each occurrence, is 0 or 1; and wherein at least one of z is 1.

In some embodiments, the radiolabeled antigen-binding protein is a compound of Formula (I):

M-L-A-[L-M_{z}]ₖ (I)

A is an anti-PSMA antibody or an anti-PSMA/anti-CD3 antigen binding molecule; L is a chelating moiety; M is a positron emitter; z is 0 or 1; and k is an integer from 0-30. In some embodiments, k is 1. In some embodiments, k is 2.

In certain embodiments, the radiolabeled antigen-binding protein is a compound of Formula (II):

A-[L-M]ₖ (II)

wherein A is an anti-PSMA antibody or anti-PSMA/anti-CD3 antigen binding molecule; L is a chelating moiety; M is a positron emitter; and k is an integer from 1-30.

In some embodiments, provided herein are compositions comprising a conjugate having the following structure:

A-Lₖ

wherein A is an anti-PSMA antibody or anti-PSMA/anti-CD3 antigen binding molecule; L is a chelating moiety; and k is an integer from 1-30; wherein the conjugate is chelated with a positron emitter in an amount sufficient to provide a specific activity suitable for clinical PET imaging.

Suitable chelating moieties, and positron emitters are provided below.

Positron Emitters and Chelating Moieties

Suitable positron emitters include, but are not limited to, those that form stable complexes with the chelating moiety and have physical half-lives suitable for immuno-PET imaging purposes. Illustrative positron emitters include, but are not limited to, ⁸⁹Zr, ⁶⁸Ga, ⁶⁴Cu, ⁴⁴Sc, and ⁸⁶Y. Suitable positron emitters also include those that directly bond with the anti-PSMA/anti-CD3 bispecific antigen binding molecule, including, but not limited to, ⁷⁶Br and ¹²⁴I, and those that are introduced via prosthetic group, e.g., ¹⁸F.

The chelating moieties described herein are chemical moieties that are covalently linked to the anti-PSMA/anti-CD3 antigen binding molecule and comprise a portion capable of chelating a positron emitter, i.e., capable of reacting with a positron emitter to form a coordinated chelate complex. Suitable moieties include those that allow efficient loading of the particular metal and form metal-chelator complexes that are sufficiently stable in vivo for diagnostic uses, e.g., immuno-PET imaging. Illustrative chelating moieties include those that minimize dissociation of the positron emitter and accumulation in mineral bone, plasma proteins, and/or bone marrow depositing to an extent suitable for diagnostic uses.

Examples of chelating moieties include, but are not limited to, those that form stable complexes with positron emitters ⁸⁹Zr, ⁶⁸Ga, ⁶⁴Cu, ⁴⁴Sc, and/or ⁸⁶Y. Illustrative chelating moieties include, but are not limited to, those described in Nature Protocols, 5(4): 739, 2010; Bioconjugate Chem., 26(12): 2579 (2015); Chem Commun (Camb), 51(12): 2301 (2015); Mol. Pharmaceutics, 12: 2142 (2015); Mol. Imaging Biol., 18: 344 (2015); Eur. J. Nucl. Med. Mol. Imaging, 37:250 (2010); Eur. J. Nucl. Med. Mol. Imaging (2016). doi:10.1007/s00259-016-3499-x; Bioconjugate Chem., 26(12): 2579 (2015); WO 2015/140212A1; and U.S. Pat. No. 5,639,879, incorporated by reference in their entireties.

Illustrative chelating moieties also include, but are not limited to, those that comprise desferrioxamine (DFO), 1,4,7,10-tetraacetic acid (DOTA), diethylenetriaminepentaacetic acid (DTPA), ethylenediaminetetraacetic acid (EDTA), (1,4,7,10-Tetraazacyclododecane-1,4,7,10-tetra(methylene phosphonic) acid (DOTP), 1R, 4R, 7R, 10R)-α′α″α′″-Tetramethyl-1,4,7,10-tetraazacyclododecane-1,4,7,10-tetraacetic acid (DOTMA), 1,4,8,11-Tetraazacyclotetradecane-1,4,8,11-tetraacetic acid (TETA), H₄octapa, H₆phospa, H₂dedpa, H₆decapa, H₂azapa, HOPO, DO2A, 1,4,7,10-Tetrakis(carbamoylmethyl)-1,4,7,10-tetraazacyclododecane (DOTAM), 1,4,7-triazacyclononane-N,N′,N″-triacetic acid (NOTA), 1,4,7,10-Tetrakis(carbamoylmethyl)-1,4,7,10-tetraazacyclododecane (DOTAM), 1,4,8,11-tetraazabicyclo[6.6.2]hexadecane-4,11-dicetic acid (CB-TE2A), 1,4,7,10-Tetraazacyclododecane (Cyclen), 1,4,8,11-Tetraazacyclotetradecane (Cyclam), octadentate chelators, octadentate bifunctional chelating agents, e.g., DFO*, hexadentate chelators, phosphonate-based chelators, macrocyclic chelators, chelators comprising macrocyclic terephthalamide ligands, bifunctional chelators, fusarinine C and fusarinine C derivative chelators, triacetylfusarinine C (TAFC), ferrioxamine E (FOXE), ferrioxamine B (FOXB), ferrichrome A (FCHA), and the like.

In some embodiments, the chelating moieties are covalently bonded to the anti-PSMA/anti-CD3 bispecific antigen binding molecule, via a linker moiety, which covalently attaches the chelating portion of the chelating moiety to the binding protein. In some embodiments, these linker moieties are formed from a reaction between a reactive moiety of the bispecific antigen binding molecule, e.g., cysteine or lysine of an antibody, and reactive moiety that is attached to a chelator, including, for example, a p-isothiocyanatobenyl group and the reactive moieties provided in the conjugation methods below. In addition, such linker moieties optionally comprise chemical groups used for purposes of adjusting polarity, solubility, steric interactions, rigidity, and/or the length between the chelating portion and the anti-PSMA/anti-CD3 bispecific antigen binding molecule.

Preparation of Radiolabeled Anti-PSMA/Anti-CD3 Bispecific Antigen Binding Molecule Conjugates

The radiolabeled anti-PSMA antibody conjugates and anti-PSMA/anti-CD3 bispecific antigen binding molecule conjugates can be prepared by (1) reacting the antigen binding molecule with a molecule comprising a positron emitter chelator and a moiety reactive to the desirable conjugation site of the bispecific binding protein and (2) loading the desirable positron emitter.

Suitable conjugation sites include, but are not limited to, lysine and cysteine, both of which can be, for example, native or engineered, and can be, for example, present on the heavy or light chain of an antibody. Cysteine conjugation sites include, but are not limited to, those obtained from mutation, insertion, or reduction of antibody disulfide bonds. Methods for making cysteine engineered antibodies include, but are not limited to, those disclosed in WO2011/056983. Site-specific conjugation methods can also be used to direct the conjugation reaction to specific sites of an antibody, achieve desirable stoichiometry, and/or achieve desirable chelator-to-antibody ratios. Such conjugation methods are known to those of ordinary skill in the art and include, but are not limited to cysteine engineering and enzymatic and chemo-enzymatic methods, including, but not limited to, glutamine conjugation, Q295 conjugation, and transglutaminase-mediated conjugation, as well as those described in J. Clin. Immunol., 36: 100 (2016), incorporated herein by reference in its entirety. Suitable moieties reactive to the desirable conjugation site generally enable efficient and facile coupling of the anti-PSMA/anti-CD3 bispecific antigen binding molecule, e.g., bispecific antibody and positron emitter chelator. Moieties reactive to lysine and cysteine sites include electrophilic groups, which are known to those of ordinary skill. In certain aspects, when the desired conjugation site is lysine, the reactive moiety is an isothiocyanate, e.g., p-isothiocyanatobenyl group or reactive ester. In certain aspects, when the desired conjugation site is cysteine, the reactive moiety is a maleimide.

When the chelator is desferrioxamine (DFO), suitable reactive moieties include, but are not limited to, an isothiocyantatobenzyl group, an n-hydroxysuccinimide ester,2,3,5,6 tetrafluorophenol ester, n-succinimidyl-S-acetylthioacetate, and those described in BioMed Research International, Vol 2014, Article ID 203601, incorporated herein by reference in its entirety. In certain embodiments, the molecule comprising a positron emitter chelator and moiety reactive to the conjugation site is p-isothiocvantatobenzvl-desferrioxamine (p-SCN-Bn-DFO):

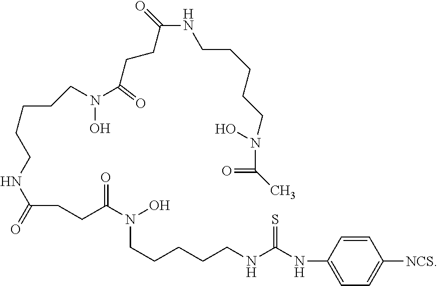

Positron emitter loading is accomplished by incubating the anti-PSMA/anti-CD3 bispecific antigen binding molecule chelator conjugate with the positron emitter for a time sufficient to allow coordination of said positron emitter to the chelator, e.g., by performing the methods described in the examples provided herein, or substantially similar method.

Illustrative Embodiments of Conjugates

Included in the instant disclosure are radiolabeled antibody conjugates comprising an anti-PSMA antibody or an anti-PSMA/anti-CD3 bispecific antigen binding molecule and a positron emitter. Also included in the instant disclosure are radiolabeled antibody conjugates comprising an anti-PSMA antibody or an anti-PSMA/anti-CD3 bispecific antigen binding molecule, a chelating moiety, and a positron emitter.

In some embodiments, the chelating moiety comprises a chelator capable of forming a complex with ⁸⁹Zr. In certain embodiments, the chelating moiety comprises desferrioxamine. In certain embodiments, the chelating moiety is p-isothiocyanatobenzyl-desferrioxamine.

In some embodiments, the positron emitter is ⁸⁹Zr. In some embodiments, less than 1.0% of the anti-PSMA antibody or anti-PSMA/anti-CD3 bispecific antigen binding molecule is conjugated with the positron emitter, less than 0.9% of the anti-PSMA antibody or anti-PSMA/anti-CD3 bispecific antigen binding molecule is conjugated with the positron emitter, less than 0.8% of the anti-PSMA antibody or anti-PSMA/anti-CD3 bispecific antigen binding molecule is conjugated with the positron emitter, less than 0.7% of the anti-PSMA antibody or anti-PSMA/anti-CD3 bispecific antigen binding molecule is conjugated with the positron emitter, less than 0.6% of the anti-PSMA antibody or anti-PSMA/anti-CD3 bispecific antigen binding molecule is conjugated with the positron emitter, less than 0.5% of the anti-PSMA antibody or anti-PSMA/anti-CD3 bispecific antigen binding molecule is conjugated with the positron emitter, less than 0.4% of the anti-PSMA antibody or anti-PSMA/anti-CD3 bispecific antigen binding molecule is conjugated with the positron emitter, less than 0.3% of the anti-PSMA antibody or anti-PSMA/anti-CD3 bispecific antigen binding molecule is conjugated with the positron emitter, less than 0.2% of the anti-PSMA antibody or anti-PSMA/anti-CD3 bispecific antigen binding molecule is conjugated with the positron emitter, or less than 0.1% of the anti-PSMA antibody or anti-PSMA/anti-CD3 bispecific antigen binding molecule is conjugated with the positron emitter.

In some embodiments, the chelating moiety-to-antibody ratio of the conjugate is from 1.0 to 2.0. As used herein, “chelating moiety-to-antibody ratio” is the average chelator moiety to antibody ratio and is a measure of chelator load per antibody. This ratio is analogous to “DAR”, i.e., drug-antibody ratio, which is used by those skilled in the art to measure drug load per antibody for antibody-drug conjugates (ADCs); in the context of the conjugates described herein for iPET imaging, the chelating moiety-to-antibody ratio can be ascertained using methods described herein and others known in the art for the determination of DAR, e.g. those described in Wang et al., Antibody-Drug Conjugates, The 21^{st} Century Magic Bullets for Cancer (2015). In some embodiments, the chelating moiety-to-antibody ratio is about 1.7. In some embodiments, the chelating moiety-to-antibody ratio is from 1.0 to 2.0. In some embodiments, the chelating moiety-to-antibody ratio is about 1.7.

In a particular embodiment, the chelating moiety is p-isothiocyanatobenzyl-desferrioxamine and the positron emitter is ⁸⁹Zr. In another particular embodiment, the chelating moiety is p-isothiocyanatobenzyl-desferrioxamine and the positron emitter is ⁸⁹Zr, and the chelating moiety-to-antibody ratio of the conjugate is from 1 to 2.

In some embodiments, provided herein are anti-PSMA antibodies or anti-PSMA/anti-CD3 bispecific antigen binding molecules, wherein said antigen-binding molecules are covalently bonded to one or more moieties having the following structure:

-L-M_{z}

wherein L is a chelating moiety; M is a positron emitter; and z, independently at each occurrence, is 0 or 1; and wherein at least one of z is 1. In certain embodiments, the radiolabeled antigen-binding protein is a compound of Formula (I):

M-L-A-[L-M_{z}]ₖ (I)

A is an anti-PSMA antibody or an anti-PSMA/anti-CD3 bispecific antigen binding molecule; L is a chelating moiety; M is a positron emitter; z is 0 or 1; and k is an integer from 0-30. In some embodiments, k is 1. In some embodiments, k is 2.

In some embodiments, L is:

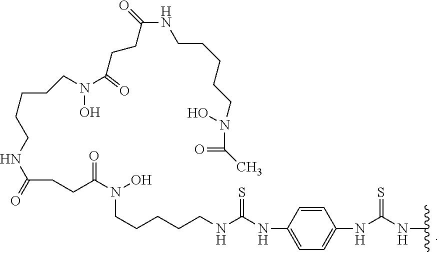 [SMILES: *NC(=S)Nc1ccc(NC(=S)NCCCCCN(O)C(=O)CCC(=O)NCCCCCN(O)C(=O)CCC(=O)NCCCCCN(O)C(C)=O)cc1]

In some embodiments, M is ⁸⁹Zr.

In some embodiments, k is an integer from 1 to 2. In some embodiments, k is 1. In some embodiments, k is 2.

In some embodiments, -L-M is

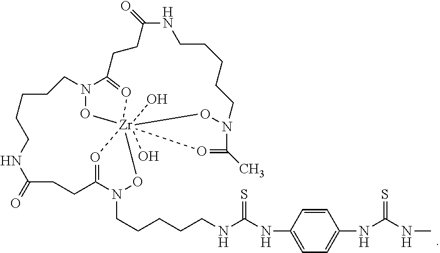

Included in the instant disclosure are also methods of synthesizing a radiolabeled antibody conjugate comprising contacting a compound of Formula (III):

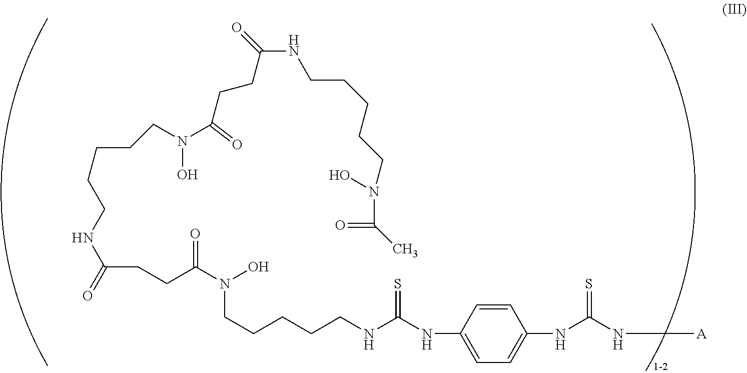

with ⁸⁹Zr, wherein A is an anti-PSMA antibody or an anti-PSMA/anti-CD3 bispecific antigen binding molecule. In certain embodiments, the compound of Formula (III) is synthesized by contacting the anti-PSMA antibody or the anti-PSMA/anti-CD3 bispecific antigen binding molecule with p-SCN-Bn-DFO.

Provided herein is also the product of the reaction between a compound of Formula (III) with ⁸⁹Zr.

Provided herein are compounds of Formula (III):

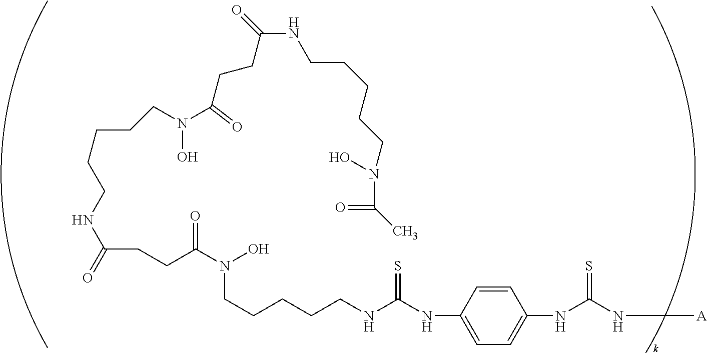

wherein A is an anti-PSMA/anti-CD3 bispecific antigen binding molecule and k is an integer from 1-30. In some embodiments, k is 1 or 2.

Provided herein are antibody conjugates comprising (i) an anti-PSMA antibody or an anti-PSMA/anti-CD3 bispecific antigen binding molecule and (ii) one or more chelating moieties.

In some embodiments, the chelating moiety comprises:

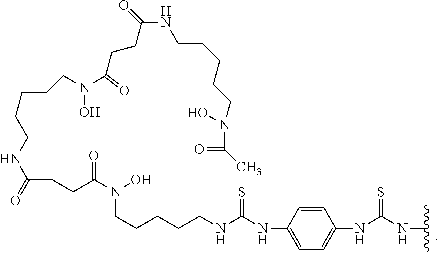 [SMILES: *NC(=S)Nc1ccc(NC(=S)NCCCCCN(O)C(=O)CCC(=O)NCCCCCN(O)C(=O)CCC(=O)NCCCCCN(O)C(C)=O)cc1]

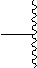

is a covalent bond to the antibody or antigen-binding fragment thereof.

In some aspects, the antibody conjugate has a chelating moiety-to-antibody ratio of from about 1.0 to about 2.0. In some aspects, the antibody conjugate has a chelating moiety-to-antibody ratio of about 1.7.

In some embodiments, provided herein are compositions comprising a conjugate having the following structure:

A-Lₖ

wherein A is an anti-PSMA antibody or an anti-PSMA/anti-CD3 bispecific antigen binding molecule; L is a chelating moiety; and k is an integer from 1-30; the conjugate is chelated with a positron emitter in an amount sufficient to provide a specific activity suitable for clinical PET imaging. In some embodiments, the amount of chelated positron emitter is an amount sufficient to provide a specific activity of about 1 to about 50 mCi per 1-50 mg of the anti-PSMA/anti-CD3 bispecific antigen binding molecule.

In some embodiments, the amount of chelated positron emitter is an amount sufficient to provide a specific activity of up to 50 mCi, up to 45 mCi, up to 40 mCi, up to 35 mCi, up to 30 mCi, up to 25 mCi, or up to 10 mCi per 1-50 mg of the anti-PSMA/anti-CD3 bispecific antigen binding molecule, for example, in a range of about 5 to about 50 mCi, about 10 to about 40 mCi, about 15 to about 30 mCi, about 7 to about 25 mCi, about 20 to about 50 mCi, or about 5 to about 10 mCi.

Methods of Using Radiolabeled Immunoconjugates

In certain aspects, the present disclosure provides diagnostic and therapeutic methods of use of the radiolabeled antibody conjugates of the present disclosure.

According to one aspect, the present disclosure provides methods of detecting PSMA in a tissue, the methods comprising administering a radiolabeled anti-PSMA antibody conjugate or anti-PSMA/anti-CD3 bispecific antigen binding molecule conjugate provided herein to the tissue; and visualizing the PSMA expression by positron emission tomography (PET) imaging. In certain embodiments, the tissue comprises cells or cell lines. In certain embodiments, the tissue is present in a subject, wherein the subject is a mammal. In certain embodiments, the subject is a human subject. In certain embodiments, the subject has a disease or disorder selected from the group consisting of cancer that expresses the PSMA antigen such as prostate cancer, kidney cancer, bladder cancer, colorectal cancer, and gastric cancer. In one embodiment, the subject has prostate cancer.

According to one aspect, the present disclosure provides methods of imaging a tissue that expresses PSMA comprising administering a radiolabeled anti-PSMA antibody conjugate or anti-PSMA/anti-CD3 bispecific antigen binding molecule conjugate of the present disclosure to the tissue; and visualizing the PSMA expression by positron emission tomography (PET) imaging. In one embodiment, the tissue is comprised in a tumor. In one embodiment, the tissue is comprised in a tumor cell culture or tumor cell line. In one embodiment, the tissue is comprised in a tumor lesion in a subject. In one embodiment, the tissue is intratumoral lymphocytes in a tissue. In one embodiment, the tissue comprises PSMA-expressing cells.

According to one aspect, the present disclosure provides methods for determining if a subject having a tumor is suitable for anti-tumor therapy, the methods comprising administering a radiolabeled antibody conjugate of the present disclosure, and localizing the administered radiolabeled antibody conjugate in the tumor by PET imaging wherein presence of the radiolabeled antibody conjugate in the tumor identifies the subject as suitable for anti-tumor therapy.

According to one aspect, the present disclosure provides methods for predicting response of a subject having a solid tumor to an anti-tumor therapy, the methods comprising determining if the tumor is PSMA positive, wherein a positive response of the subject is predicted if the tumor is PSMA positive. In certain embodiments, the tumor is determined positive by administering a radiolabeled antibody conjugate of the present disclosure and localizing the radiolabeled antibody conjugate in the tumor by PET imaging wherein presence of the radiolabeled antibody conjugate in the tumor indicates that the tumor is PSMA positive.

According to one aspect, the present disclosure provides methods for detecting a PSMA positive tumor in a subject. The methods, according to this aspect, comprise administering a radiolabeled antibody conjugate of the present disclosure to the subject; and determining localization of the radiolabeled antibody conjugate by PET imaging, wherein presence of the radiolabeled antibody conjugate in a tumor indicates that the tumor is PSMA positive.

Provided herein are methods for predicting a positive response to an anti-tumor therapy comprising: administering a radiolabeled anti-PSMA antibody conjugate or anti-PSMA/anti-CD3 bispecific antigen binding molecule conjugate to the subject to determine the presence of PSMA positive cells in the solid tumor. The presence of PSMA-positive cells predicts a positive response to an anti-tumor therapy.

As used herein, the expression “a subject in need thereof” means a human or non-human mammal that exhibits one or more symptoms or indications of cancer, and/or who has been diagnosed with cancer, including a solid tumor and who needs treatment for the same. In many embodiments, the term “subject” may be interchangeably used with the term “patient”. For example, a human subject may be diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker (e.g., CA125). The expression includes subjects with primary or established tumors. In specific embodiments, the expression includes human subjects that have and/or need treatment for a solid tumor, e.g., colon cancer, breast cancer, lung cancer, prostate cancer, skin cancer, liver cancer, bone cancer, ovarian cancer, cervical cancer, pancreatic cancer, head and neck cancer, and brain cancer. The term includes subjects with primary or metastatic tumors (advanced malignancies). In certain embodiments, the expression “a subject in need thereof” includes subjects with a solid tumor that is resistant to or refractory to or is inadequately controlled by prior therapy (e.g., treatment with an anti-cancer agent). For example, the expression includes subjects who have been treated with one or more lines of prior therapy such as treatment with chemotherapy (e.g., carboplatin or docetaxel). In certain embodiments, the expression “a subject in need thereof” includes subjects with a solid tumor which has been treated with one or more lines of prior therapy but which has subsequently relapsed or metastasized. In certain embodiments, the methods of the present disclosure are used in a subject with a solid tumor. The terms “tumor”, “cancer” and “malignancy” are interchangeably used herein. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and lymphomas.

In certain embodiments, the cancer or tumor is a selected from the group consisting of astrocytoma, anal cancer, bladder cancer, blood cancer, blood cancer, bone cancer, brain cancer, breast cancer, cervical cancer, clear cell renal cell carcinoma, colorectal cancer, microsatellite-intermediate colorectal cancer, cutaneous squamous cell carcinoma, diffuse large B-cell lymphoma, endometrial cancer, esophageal cancer, fibrosarcoma, gastric cancer, glioblastoma, glioblastoma multiforme, head and neck squamous cell carcinoma, hepatic cell carcinoma, leukemia, liver cancer, leiomyosarcoma, lung cancer, lymphoma, melanoma, mesothelioma, myeloma, nasopharyngeal cancer, non-small cell lung cancer, osteosarcoma, ovarian cancer, pancreatic cancer, primary and/or recurrent cancer, prostate cancer, renal cell carcinoma, rhabdomyosarcoma, salivary gland cancer, skin cancer, small cell lung cancer, squamous cell cancer, stomach cancer, synovial sarcoma, testicular cancer, thyroid cancer, triple negative breast cancer, uterine cancer, and Wilms' tumor. In some aspects, the cancer is a primary cancer. In some aspects, the cancer is metastatic and/or recurrent cancer.

In certain embodiments, the cancer or tumor is selected from a PSMA positive tumor, such as a tumor originating in prostatic epithelium, duodenal mucosa, proximal renal tubules, or colonic crypt neuroendocrine cells. In some aspects, the cancer is bladder cancer, renal cancer, gastric cancer, or colorectal carcinoma. In some aspects, the cancer is prostate cancer. In some aspects, the cancer is metastatic cancer originating from a primary prostate tumor.

As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, and/or to increase duration of survival of the subject.

In certain embodiments, the radiolabeled anti-PSMA antibody conjugate or anti-PSMA/anti-CD3 bispecific antigen binding molecule conjugate is administered intravenously or subcutaneously to the subject. In certain embodiments, the radiolabeled antibody conjugate is administered intra-tumorally. Upon administration, the radiolabeled antibody conjugate is localized in the tumor. The localized radiolabeled antibody conjugate is imaged by PET imaging and the uptake of the radiolabeled antibody conjugate by the tumor is measured by methods known in the art. In certain embodiments, the imaging is carried out 1, 2, 3, 4, 5, 6 or 7 days after administration of the radiolabeled conjugate. In certain embodiments, the imaging is carried out on the same day upon administration of the radiolabeled antibody conjugate.

In certain embodiments, the radiolabeled anti-PSMA antibody conjugate or anti-PSMA/anti-CD3 bispecific antigen binding molecule conjugate can be administered at a dose of about 0.1 mg/kg of body weight to about 100 mg/kg of body weight of the subject, for example, about 0.1 mg/kg to about 50 mg/kg, or about 0.5 mg/kg to about 25 mg/kg, or about 0.1 mg/kg to about 1.0 mg/kg of body weight.

Therapeutic Formulation and Administration

Useful according to the present disclosure are pharmaceutical compositions comprising the antigen-binding molecules useful herein. In some aspects, the pharmaceutical composition further comprises an anti-4-1BB agonist. The pharmaceutical compositions are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding molecule administered to a patient may vary depending upon the age and the size of the patient, target disease, conditions, route of administration, and the like. The preferred dose is typically calculated according to body weight or body surface area. When a bispecific antigen-binding molecule is used for therapeutic purposes in an adult patient, it may be advantageous to intravenously administer the bispecific antigen-binding molecule normally at a single dose of about 0.01 to about 20 mg/kg body weight, more preferably about 0.02 to about 7, about 0.03 to about 5, or about 0.05 to about 3 mg/kg body weight. In some aspects, it may be advantageous to intravenously administer the bispecific antigen-binding molecule normally at a single dose of about 50 mg, or about 75 mg, or about 100 mg, or about 150 mg, or about 200 mg, or about 250 mg, or about 300 mg, or about 350 mg, or about 400 mg. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

The dose of anti-4-1BB agonist administered to a patient may vary depending upon the age and the size of the patient, target disease, conditions, route of administration, and the like. The preferred dose is typically calculated according to body weight or body surface area. When an anti-4-1BB agonist is used for therapeutic purposes in an adult patient, it may be advantageous to intravenously administer the agonist normally at a single dose of about 0.01 to about 20 mg/kg body weight, more preferably about 0.02 to about 7, about 0.03 to about 5, or about 0.05 to about 3, or about 2.5 mg/kg body weight.

Various delivery systems are known and can be used to administer the pharmaceutical composition useful herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition useful herein can be delivered subcutaneously or intravenously with a standard needle and syringe. In addition, with respect to subcutaneous delivery, a pen delivery device readily has applications in delivering a pharmaceutical composition useful herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition useful herein. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition useful herein include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.5 to about 500 mg per dosage form in a unit dose; especially in the form of injection, it is preferred that the aforesaid antibody is contained in about 5 to about 100 mg and in about 10 to about 250 mg for the other dosage forms.

Combination Therapies and Formulations

The present disclosure provides methods which comprise administering a pharmaceutical composition comprising any of the exemplary monospecific or bispecific antigen-binding molecules described herein in combination with an anti-4-1BB agonist, and one or more additional therapeutic agents. Exemplary additional therapeutic agents that may be combined with or administered in combination with an anti-4-1BB agonist and a bispecific antigen-binding molecule useful herein include, e.g., an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2, anti-ErbB3 or anti-ErbB4 antibody or small molecule inhibitor of ErbB2, ErbB3 or ErbB4 activity), an antagonist of EGFRvIII (e.g., an antibody that specifically binds EGFRvIII), a cMET anagonist (e.g., an anti-cMET antibody), an IGF1R antagonist (e.g., an anti-IGF1R antibody), a B-raf inhibitor (e.g., vemurafenib, sorafenib, GDC-0879, PLX-4720), a PDGFR-α inhibitor (e.g., an anti-PDGFR-α antibody), a PDGFR-β inhibitor (e.g., an anti-PDGFR-β antibody), a VEGF antagonist (e.g., a VEGF-Trap, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), a FOLH1 (PSMA) antagonist, a PRLR antagonist (e.g., an anti-PRLR antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin antibody), etc. Other agents that may be beneficially administered in combination with the compositions provided herein include cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors. The pharmaceutical compositions useful herein (e.g., pharmaceutical compositions comprising an anti-CD3/anti-PSMA bispecific antigen-binding molecule as disclosed herein) may also be administered as part of a therapeutic regimen comprising an anti-4-1BB agonist and one or more therapeutic combinations selected from “ICE”: ifosfamide (e.g., Ifex®), carboplatin (e.g., Paraplatin®), etoposide (e.g., Etopophos®, Toposar®, VePesid®, VP-16); “DHAP”: dexamethasone (e.g., Decadron®), cytarabine (e.g., Cytosar-U®, cytosine arabinoside, ara-C), cisplatin (e.g., Platinol®-AQ); and “ESHAP”: etoposide (e.g., Etopophos®, Toposar®, VePesid®, VP-16), methylprednisolone (e.g., Medrol®), high-dose cytarabine, cisplatin (e.g., Platinol®-AQ).

The present disclosure also includes therapeutic combinations comprising any of the antigen-binding molecules mentioned herein and an inhibitor of one or more of VEGF, Ang2, DLL4, EGFR, ErbB2, ErbB3, ErbB4, EGFRvIII, cMet, IGF1R, B-raf, PDGFR-α, PDGFR-β, PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody or an antibody fragment (e.g., Fab fragment; F(ab′)2 fragment; Fd fragment; Fv fragment; scFv; dAb fragment; or other engineered molecules, such as diabodies, triabodies, tetrabodies, minibodies and minimal recognition units). The antigen-binding molecules disclosed herein may also be administered and/or co-formulated in combination with antivirals, antibiotics, analgesics, corticosteroids and/or NSAIDs. The antigen-binding molecules disclosed herein may also be administered as part of a treatment regimen that also includes radiation treatment and/or conventional chemotherapy.

The additional therapeutically active component(s) may be administered just prior to, concurrent with, or shortly after the administration of an antigen-binding molecule useful herein; (for purposes of the present disclosure, such administration regimens are considered the administration of an antigen-binding molecule “in combination with” an additional therapeutically active component).

The present disclosure includes pharmaceutical compositions in which an antigen-binding molecule useful herein is co-formulated with one or more of the additional therapeutically active component(s) as described elsewhere herein.

Administration Regimens

According to certain embodiments of the present disclosure, multiple doses of an antigen-binding molecule (e.g., an anti-PSMA antibody or an anti-CD3/anti-PSMA bispecific antigen-binding molecule) may be administered to a subject over a defined time course. In addition, multiple doses of an anti-4-1BB agonist may be administered to a subject over a defined time course. The methods according to this aspect comprise sequentially administering to a subject one or more doses of each therapeutic, i.e. one or more doses of an antigen-binding molecule and one or more doses of an anti-4-1BB agonist. As used herein, “sequentially administering” means that each dose of a therapeutic, e.g., an antigen-binding molecule, is administered to the subject at a different point in time, e.g., on different days separated by a predetermined interval (e.g., hours, days, weeks or months). The present disclosure includes methods which comprise sequentially administering to the patient a single initial dose of an antigen-binding molecule, referred to as a loading dose, followed by one or more secondary doses of the antigen-binding molecule, and optionally followed by one or more tertiary doses of the antigen-binding molecule. The present disclosure includes methods which comprise sequentially administering to the patient a single initial dose of an anti-4-1BB agonist, referred to as a loading dose, followed by one or more secondary doses of the anti-4-1BB agonist, and optionally followed by one or more tertiary doses of the anti-4-1BB agonist.

The terms “initial dose,” “secondary doses,” and “tertiary doses,” refer to the temporal sequence of administration of the antigen-binding molecule and/or anti-4-1BB agonist useful herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antigen-binding molecule (or anti-4-1BB agonist), but generally may differ from one another in terms of frequency of administration. In certain embodiments, however, the amount of an antigen-binding molecule (or anti-4-1BB agonist) contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antigen-binding molecule (or anti-4-1BB agonist) which is administered to a patient prior to the administration of the very next dose in the sequence with no intervening doses.

The methods according to this aspect of the disclosure may comprise administering to a patient any number of secondary and/or tertiary doses of an anti-4-1BB agonist, an anti-PSMA antibody, or a bispecific antigen-binding molecule that specifically binds PSMA and CD3. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Diagnostic Uses of the Antibodies

The bispecific antibodies of the present disclosure may also be used to detect and/or measure PSMA, or PSMA-expressing cells in a sample, e.g., for diagnostic purposes. For example, an anti-PSMA antibody, or fragment thereof, may be used to diagnose a condition or disease characterized by aberrant expression (e.g., over-expression, under-expression, lack of expression, etc.) of PSMA. Exemplary diagnostic assays for PSMA may comprise, e.g., contacting a sample, obtained from a patient, with an anti-PSMAxCD3 bispecific antibody, wherein the bispecific antibody is labeled with a detectable label or reporter molecule. Alternatively, an unlabeled anti-PSMAxCD3 bispecific antibody can be used in diagnostic applications in combination with a secondary antibody which is itself detectably labeled. The detectable label or reporter molecule can be a radioisotope, such as ³H, ¹⁴O ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Another exemplary diagnostic use of the anti-PSMAxCD3 bispecific antibodies useful herein includes ⁸⁹Zr-labeled, such as ⁸⁹Zr-desferrioxamine-labeled, antibody for the purpose of noninvasive identification and tracking of tumor cells in a subject (e.g. positron emission tomography (PET) imaging). (See, e.g., Tavare, R. et al. Cancer Res. 2016 Jan. 1; 76(1):73-82; and Azad, B B. et al. Oncotarget. 2016 Mar. 15; 7(11):12344-58.) Specific exemplary assays that can be used to detect or measure PSMA in a sample include enzyme-linked immunosorbent assay (ELISA), radioimmunoassay (RIA), and fluorescence-activated cell sorting (FACS).

Samples that can be used in PSMA diagnostic assays according to the present disclosure include any tissue or fluid sample obtainable from a patient which contains detectable quantities of PSMA protein, or fragments thereof, under normal or pathological conditions. Generally, levels of PSMA in a particular sample obtained from a healthy patient (e.g., a patient not afflicted with a disease or condition associated with abnormal PSMA levels or activity) will be measured to initially establish a baseline, or standard, level of PSMA. This baseline level of PSMA can then be compared against the levels of PSMA measured in samples obtained from individuals suspected of having a PSMA related disease (e.g., a tumor containing PSMA-expressing cells) or condition.

### EXAMPLES

The following examples are put forth so as to provide those of ordinary skill in the art with a complete disclosure and description of how to make and use the methods and compositions useful herein, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Bispecific Antibodies that Bind Prostate-Specific Membrane Antigen (PSMA) and CD3

The present disclosure provides anti-PSMA antibodies useful according to the methods disclosed herein. The antibodies were generated according to the disclosure provided in U.S. Pat. No. 10,179,819. Exemplary antibodies useful herein include the H1H11810P antibody, and the CDR, HCVR, and LCVR sequences encompassed by this antibody. As such, an exemplary anti-PSMA antibody or antigen-binding fragment thereof comprises an HCVR of SEQ ID NO: 66 and an LCVR of SEQ ID NO: 1386 as disclosed in U.S. Pat. No. 10,179,819.

The present disclosure also provides bispecific antigen-binding molecules that bind CD3 and Prostate-Specific Membrane Antigen (PSMA); such bispecific antigen-binding molecules are also referred to herein as “anti-PSMA/anti-CD3 bispecific molecules.” The anti-PSMA portion of the anti-PSMA/anti-CD3 bispecific molecule is useful for targeting tumor cells that express PSMA, and the anti-CD3 portion of the bispecific molecule is useful for activating T-cells. The simultaneous binding of PSMA on a tumor cell and CD3 on a T-cell facilitates directed killing (cell lysis) of the targeted tumor cell by the activated T-cell.

Bispecific antibodies comprising an anti-PSMA-specific binding domain and an anti-CD3-specific binding domain were constructed using standard methodologies, wherein the anti-PSMA antigen binding domain and the anti-CD3 antigen binding domain each comprise different, distinct HCVRs paired with a common LCVR. In some instances the bispecific antibodies were constructed utilizing a heavy chain from an anti-CD3 antibody, a heavy chain from an anti-PSMA antibody and a common light chain. In other instances, the bispecific antibodies were constructed utilizing a heavy chain from an anti-CD3 antibody, a heavy chain from an anti-PSMA antibody and a light chain from an anti-CD3 antibody. In some instances, the bispecific antibodies were constructed utilizing a HCVR from an anti-CD3 antibody, a HCVR from an anti-PSMA antibody and a common LCVR. In other instances, the bispecific antibodies were constructed utilizing a HCVR from an anti-CD3 antibody, a HCVR from an anti-PSMA antibody, and a LCVR from an anti-CD3 antibody.

A summary of the component parts of an exemplary anti-PSMAxCD3 bispecific antibody construct is set forth in Table 1.

TABLE 1
Anti-PSMAxCD3 Bispecific Antigen Binding Molecule

| PSMA/CD3- | PSMA-binding | CD3-binding arm | Common light |
|---|---|---|---|
| 005 | arm heavy chain | heavy chain | chain |
| VRs | SEQ ID NO: 1 | SEQ ID NO: 2 | SEQ ID NO: 3 |

### Example 2: PSMA-Targeting CD3-Bispecific Induces Anti-Tumor Responses which are Enhanced by 4-1BB Co-Stimulation

PSMAxCD3 bispecific antibody targeting prostate cancer tumor antigen, PSMA, was evaluated in several preclinical solid tumor models. Mice humanized for CD3 and PSMA were developed to examine anti-tumor efficacy in the presence of an intact immune system and PSMA expression in normal tissues. Immuno-PET imaging demonstrated that PSMAxCD3 accumulated in PSMA expressing tissues and tumors, associated with significant anti-tumor efficacy. However, PSMAxCD3 lost efficacy as tumor burden increased. To boost efficacy in mice with a higher tumor burden, PSMAxCD3 combined with anti-4-1BB (anti-mouse 4-1bb from InVivoPlus, isotype rat IgG1, Catalog Number BP0169) co-stimulation achieved impressive T cell activation, cytokine production, proliferation and memory, leading to enhanced efficacy and durable anti-tumor responses. This Example demonstrates that CD3-bispecific antibodies combined with anti-4-1BB co-stimulation are a viable therapeutic combination against solid tumors.

In all studies in this Example, a CD3-bispecific with an irrelevant targeting arm (CD3-binding control) was used as a control. In vivo efficacy was evaluated in both xenogeneic and syngeneic mouse models. In the xenogeneic models NSG mice were engrafted with human PBMCs and C4-2 or 22Rv1 cells (sample size 5 mice per group). For the syngeneic models (sample size 5-10 mice per group) HuT mice were implanted with TRAMP-C2-hPSMA cells. All animal studies were carried out in accordance with the Guide for the Care and Use of Laboratory Animals of the NIH.

PSMAxCD3 Induces Target-Dependent T Cell Activation and Tumor Cell Cytotoxicity

A PSMAxCD3 bispecific antigen-binding molecule was generated by immunizing VelocImmune® mice with human PSMA and CD3. The resulting PSMAxCD3-bispecific antibody is a hinge-stabilized, effector minimized, IgG4 isotype.

Flow cytometric analysis was utilized to determine binding of PSMAxCD3 to JURKAT and pre-activated human T cells, followed by detection with a PE-anti-human IgG antibody. Human T cells were pre-activated with anti-CD3/CD28 for 6 days. Post activation, 2×10⁵ activated human T cells or JURKAT cells/well were incubated for 30 minutes at 4° C. with 10 ug/ml of PSMAxCD3. After incubation, the cells were washed twice with cold PBS (1% FBS). After wash, a PE-anti-human secondary antibody was added to the cells and incubated for an additional 30 minutes. Wells containing no antibody or secondary only were used as a control. After incubation, cells were analyzed by flow cytometry on a BD FACS Canto II.

Flow cytometric analysis was also used to determine binding of PSMAxCD3 to PSMA expressing cell lines. C4-2, 22Rv1, or TRAMP-C2-hPSMA cells (2×10⁶ cells/well) were incubated with PSMAxCD3 (10 ug/ml) for 15 minutes at 4° C. Following the incubation, the cells were washed twice with cold PBS (2% FBS), and an APC-anti-human secondary antibody was added for an additional 20 minutes on ice. No staining or secondary only staining were included as controls. Samples were analyzed on a BD LSRFortessa cell analyzer.

Briefly, PSMA expressing cell lines (22Rv1 and C4-2 cells) were labeled with 1 uM of Violet Cell Tracker and plated overnight at 37° C. Separately, human PBMCs were plated in supplemented RPMI media at 1×10⁶ cells/mL and incubated overnight at 37° C. in order to enrich for lymphocytes by depleting adherent macrophages, dendritic cells, and some monocytes. The next day, target cells were co-incubated with adherent cell-depleted naïve PBMC (Effector/Target cell 4:1) and serial dilutions of either PSMAxCD3 or the CD3-binding control for 48 hours at 37° C. Cells were removed from culture plates using an enzyme free dissociation buffer, analyzed by Flow Cytometry. For FACS analysis, cells were stained with a dead/live far red cell tracker (Invitrogen). For the assessment of specificity of killing, cells were gated on Violet cell tracker labeled populations. Percent of live target cells was reported for the calculation of adjusted survival as follows: Adjusted survival=(R1/R2)*100, where R1=% live target cells in the presence of antibody, and R2=% live target cells in the absence of test antibody. T cell activation was assessed by incubating cells with directly conjugated antibodies to CD2, CD69, and CD25, and by reporting the percent of activated (CD69+) T cells or (CD25+) T cells out of total T cells (CD2+).

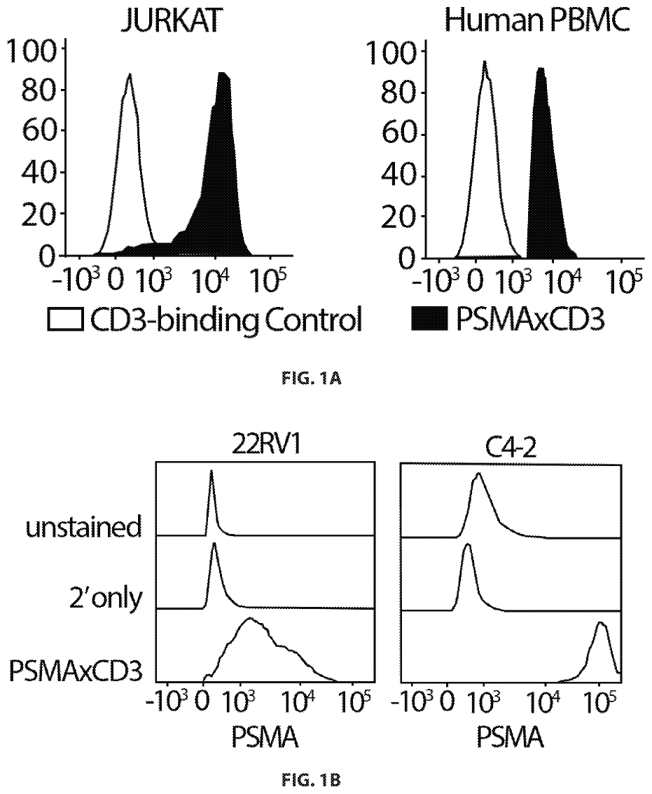

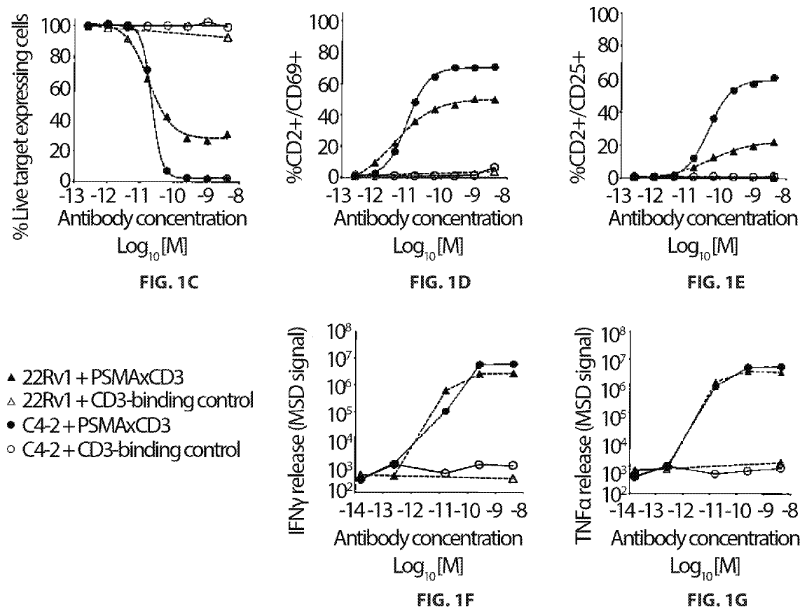

Flow cytometry analysis demonstrated that PSMAxCD3 specifically binds to CD3 on Jurkat T cells and human PBMCs (FIG. 1A). Furthermore, PSMAxCD3 specifically bound to 22Rv1 and C4-2, human tumor cells lines expressing different levels of PSMA, demonstrating that PSMAxCD3 can bind to both low and high antigen expressing cell lines (FIG. 1B). To evaluate the cytotoxic potential of PSMAxCD3, an in vitro flow cytometry-based cell-killing assay was performed. PSMAxCD3 induced the killing of 22Rv1 (EC50 1.79×10⁻¹¹) and C4-2 (EC50 2.23×10⁻¹¹) cells while the CD3-binding control did not (FIG. 1C). In response to PSMAxCD3, early activation marker CD69 (FIG. 1D) and late activation marker CD25 (FIG. 1E) were elevated on T cells. PSMAxCD3 also induced cytokine release (IFNγ and TNFα) when T cells were incubated with C4-2 or 22Rv1 tumor cells (FIG. 1F, G).

Together, these results demonstrated that PSMAxCD3 is able to induce target dependent, CD3-mediated T cell activation resulting in killing of PSMA expressing tumor cells.

PSMAxCD3 Inhibits Growth of Human Prostate Cancer Cells in a Xenogeneic Tumor Model

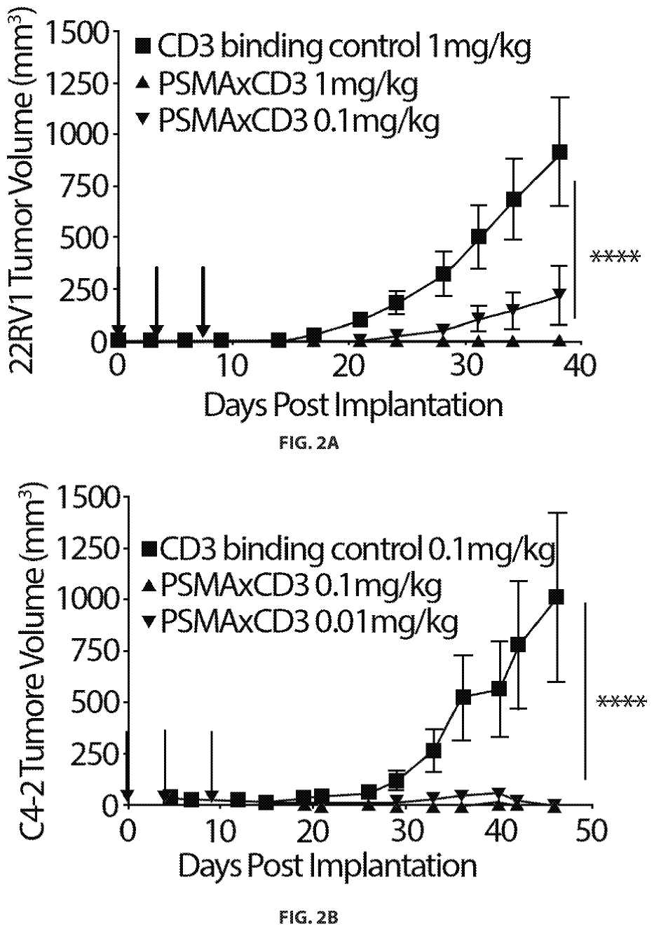

Two subcutaneous tumor xenograft mouse models were established using 22Rv1 and C4-2 human tumor cells lines. Human PBMCs were delivered as a source of human CD3 T cells in NSG mice at the time of tumor implantation and mice were treated immediately with CD3-binding control or PSMAxCD3. Mice implanted with 22Rv1 tumor cells showed tumor growth inhibition with 0.1 mg/kg and 1 mg/kg of PSMAxCD3 (FIG. 2A) while mice implanted with C4-2 tumor cells showed significant tumor growth inhibition with as low as 0.01 mg/kg of PSMAxCD3 (FIG. 2B).

Syngeneic Tumor Studies

Syngeneic studies were carried out in mice genetically modified to express human CD3 and a portion of human PSMA using VelociGene® technology. Mice (5-7/group, 8-16 weeks old) were injected with 5×10⁶ TRAMP-C2-hPSMA cells subcutaneously (SC). Mice were dosed with 5 mg/kg of PSMAxCD3 or CD3-binding control twice per week for a total of 4 treatments. Tumor growth was measured using calipers. Tumor volume based on caliper measurements were calculated by the formula: Volume=(length×width2)/2. For studies with PSMAxCD3+ anti-4-1BB (LOB12.3, BioXcell), CD3-binding control groups were treated with a Rat-IgG isotype control and anti-4-1BB groups were treated with CD3-binding control. For the tumor memory studies, mice that cleared the tumor in response to treatment were re-challenged with 1×10⁷ TRAMP-C2-hPSMA cells 35 days post tumor injection on the opposite flank.

Preparation of Immunoconjugate and Small Animal PET

A pre-calibrated Sofie Biosciences G8 PET/CT instrument (Sofie Biosciences (Culver city, CA) and Perkin Elmer) was used to acquire PET and CT images. The energy window ranged from 150 to 650 keV with a reconstructed resolution of 1.4 mm at the center of the field of view. On day 6 post dosing, mice underwent induction anesthesia using isoflurane and were kept under continuous flow of isoflurane during a 10-minute static PET acquisition followed by CT acquisition. Decay-corrected PET data and CT data were processed using VivoQuant software (inviCRO Imaging Services) into false-colored co-registered PET-CT maximum intensity projections on a color scale calibrated to indicate a signal range of 0 to 15% of injected dose per volume, expressed as % ID/g. For ex vivo biodistribution analysis, mice were euthanized following PET/CT acquisition. Blood, normal tissues and tumor were then harvested and placed into counting tubes. The γ-emission radioactivity for all samples were then counted on an automatic gamma counter (AMG, Hidex) and results reported in normalized counts per minute (cpm). The % ID for each sample was the determined using samples counts relative to dose-standards counts prepared from the original injected material. Subsequently, the individual % ID/g values were derived by dividing the % ID value by the respective weight of the appropriate blood, tissues or tumor sample.

Immuno-PET Imaging Demonstrates In Vivo Biodistribution of PSMAxCD3 in HuT Mice

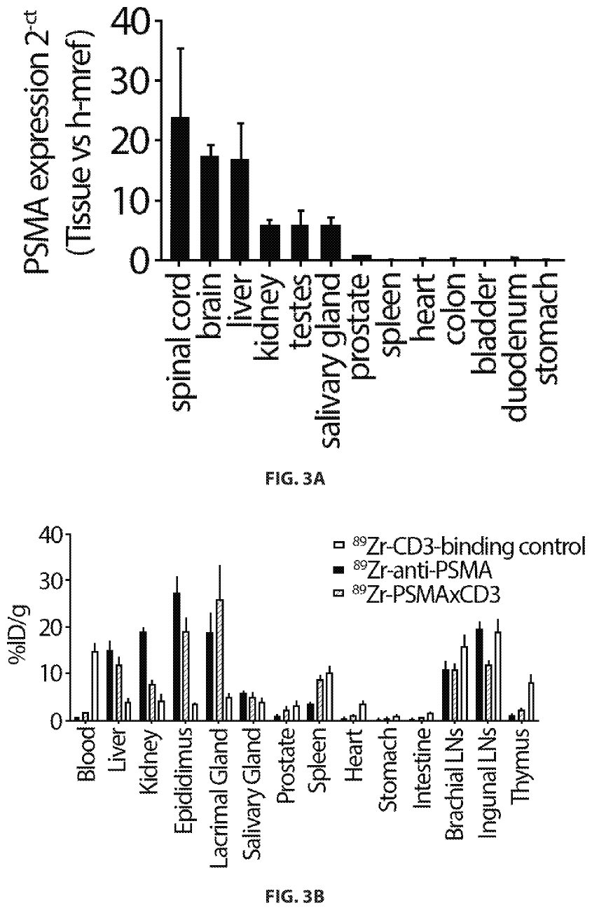

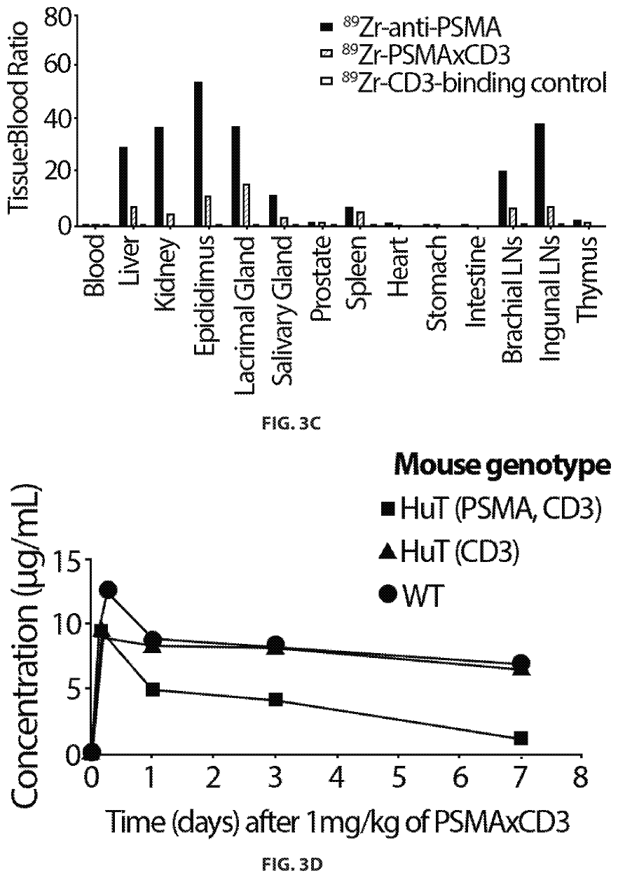

Xenogeneic models use immunodeficient mice which lack mature B, T, and NK cells. To examine PSMAxCD3 efficacy in an immunocompetent mouse model, Human Target mice (HuT) we're genetically engineered to express human PSMA and CD3 by deleting the mouse sequence and replacing it with the orthologous regions of human CD3 and PSMA. Human PSMA transcript expression was detected in the spinal cord, brain, liver, kidney, testes, and salivary glands while negligible expression was found in prostate (FIG. 3A). Additionally, PSMA protein expression was also confirmed by immunohistochemistry and showed a similar expression pattern (data not shown; and Skokos et al., submitted). To determine the in vivo bioavailability of PSMA antigen and the distribution of PSMAxCD3 in HuT mice, immuno-PET (iPET) imaging was used to track antibody localization. HuT mice were injected with ⁸⁹Zr-anti-PSMA (the bivalent antibody used to generate PSMAxCD3), ⁸⁹Zr-PSMAxCD3 or ⁸⁹Zr-CD3-binding control to evaluate tissue distribution. There was no specific targeting in mice that were injected with ⁸⁹Zr-CD3-binding control. Mice injected with ⁸⁹Zr-anti-PSMA showed specific uptake in the liver, kidneys, epididymis, lacrimal glands, salivary glands and draining lymph nodes. Of note, the brain and testes were identified as PSMA expressing tissues, however, iPET shows no targeting possibly due to the blood brain barrier and antigen inaccessibility. Mice injected with ⁸⁹Zr-PSMAxCD3 showed a similar distribution profile as the bivalent ⁸⁹Zr-anti-PSMA except for reduced uptake in the kidney and increased uptake in the spleen, indicating that distribution of PSMAxCD3 is mostly due to the PSMA-binding arm (FIGS. 3B and 3C). To confirm this, the clearance of serum drug concentrations in mice humanized for CD3 alone or in addition to PSMA was examined. While serum drug concentrations of the HuT (CD3) mice were similar to WT mice, HuT (PSMA and CD3) mice showed faster drug clearance in the serum (FIG. 3D).

Finally, humanization of these mice did not alter the polyclonal development of splenic CD8 and CD4 T cells as determined by T cell receptor (TCR) Vβ usage. HuT mice also have similar total T cell numbers and relative proportions of CD4, CD8 and regulatory T cells (Tregs) compared to WT mice. (data not shown and Crawford et al., Sci. Transl. Med. 11, eaau7534 (2019))

Together, these data demonstrated that PSMAxCD3 distribution is driven by the PSMA-binding arm and localizes to select antigen expressing tissues in our HuT mice.

PSMAxCD3 is Efficacious Against Small Established Tumors in HuT Mice

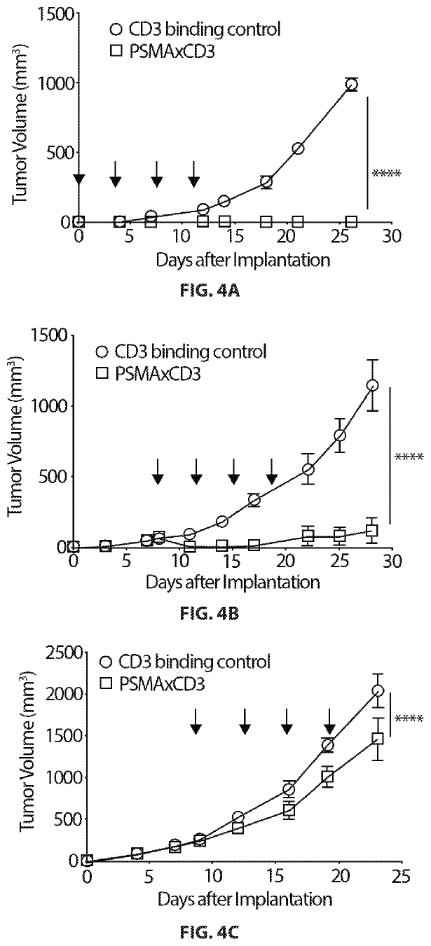

The HuT mice were subcutaneously implanted with a mouse prostate adenocarcinoma cell line expressing human PSMA (TRAMP-C2-hPSMA). PSMAxCD3 treatment initiated on the day of tumor implantation completely prevented tumor growth compared to the mice that received the CD3-binding control (FIG. 4A). PSMAxCD3 treatment initiated when tumors were approximately 50 mm³ (FIG. 4B) also demonstrated significant anti-tumor efficacy. However, despite the significant efficacy induced with these treatment regimes, when treatment was delayed until tumors were approximately 200 mm³, anti-tumor efficacy was diminished, demonstrating a brief but transient anti-tumor response (FIG. 4C). Flow cytometry confirmed that PSMA target expression was still maintained on TRAMP-C2-hPSMA tumors, indicating that the lack of efficacy was not due to the absence of target. Furthermore, a higher dose of PSMAxCD3 at 20 mg/kg was still insufficient to control 200 mm³ tumors even when PSMA target expression was maintained (data not shown).

PSMAxCD3 Targets to Tumors Regardless of Size but Efficacy is Restricted to Smaller Tumors

To determine if anti-tumor efficacy is determined by the local tumor environment or the total tumor burden in the mice, a bilateral tumor model was established so that each mouse had a small and large tumor on opposite flanks. The HuT mice were injected with 1×10⁷ (left flank) and 1.25×10⁶ (right flank) TRAMP-C2-hPSMA cells subcutaneously (SC). Mice were dosed on day 12 when the tumors measured approximately 150 mm³ (left flank) and 50 mm³ (right flank) with 5 mg/kg of PSMAxCD3 or a CD3-binding control twice per week for a total of 4 treatments.

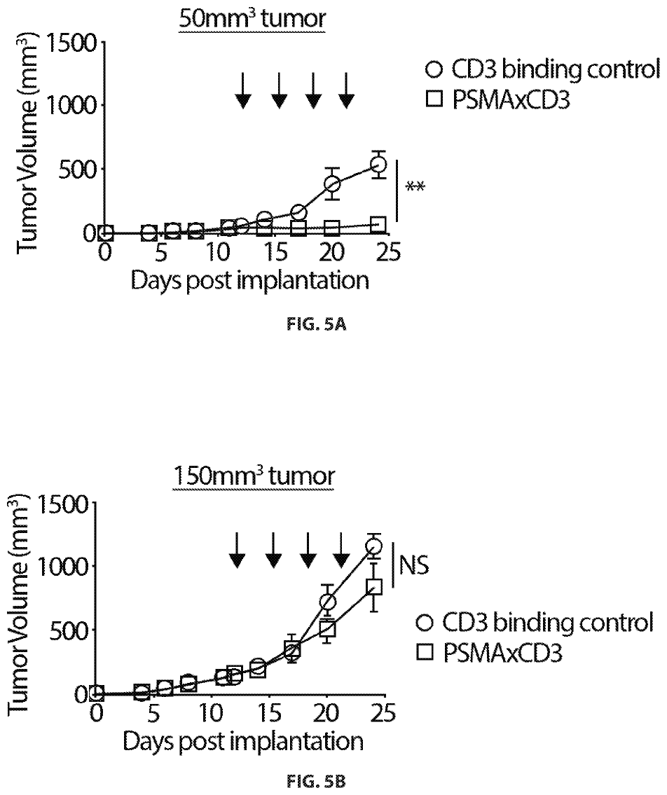

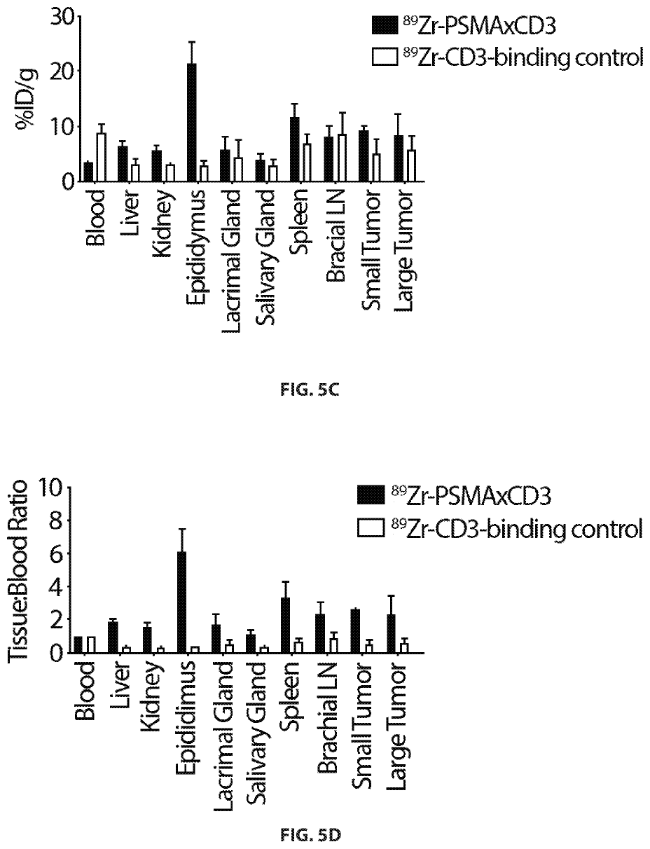

While PSMAxCD3 was able to delay tumor progression of the smaller tumors (FIG. 5A), it had no effect on the larger tumor on the opposite flank of the same animal (FIG. 5B). These findings suggested that PSMAxCD3 efficacy is determined by tumor-intrinsic factors and not by total tumor burden nor systemic T cell dysfunction. Subsequently, to determine if PSMAxCD3 can penetrate the large tumors, ⁸⁹Zr-PSMAxCD3 or ⁸⁹Zr-CD3-binding control was injected into HuT mice bearing bilateral tumors. Mice injected showed specific uptake of ⁸⁹Zr-PSMAxCD3 in peripheral tissues and tumors. In contrast, mice did not show specific uptake of ⁸⁹Zr-CD3-binding control in the tumors or tissues. Furthermore, ex vivo biodistribution analysis confirmed that there is similar uptake of PSMAxCD3 between the small and large tumors, therefore the lack of response is not due to the absence of PSMAxCD3 targeting (FIGS. 5C and 5D).

Ex Vivo Flow Cytometry:

Flow cytometry was used to detect T cells in the circulation and to examine the activation status of intratumoral T cells 48 hrs or 96 hrs after treatment, or to examine PSMA target maintenance on tumor cells. Tumors were mechanically disrupted and digested for 9 minutes at 42° C. in the presence of collagenase II (175 units/mL; Worthington), collagenase IV (200 units/mL; Gibco), and DNase 1 (400 units/mL; Sigma). The digested material was then passed through a cell strainer. To detect T cells, the combination of CD45 (30-F11, Biolegend), CD90.2 (30-H12, Biolegend), CD8 (53-6.7 BD Pharmingen), CD4 (GK1.5, BD Pharmingen) and FOXP3 (FJK-165, EBiosciences) was used. T cell activation was examined using antibodies to Granzyme B (GB11, BD Pharmingen), Ki67 (16A8, Biolegend) and 4-1BB (IAH2, BD Pharmingen). Staining was carried out using the Ebioscience FoxP3 staining buffer set. T cells were identified as CD45+, CD90.2+, CD8+, CD4+ or CD4+ FOXP3+.

PSMAxCD3 Induces T Cell Infiltration and Activation in Small and Large Tumors

To assess the frequency and spatial distribution of intratumoral T cells, tumors were analyzed by immunohistochemistry. 5 μm paraffin sections of tissues or tumors stained with an either anti-PSMA (ERP6253, ABCAM), anti-CD3 (A045229, DAKO), anti-CD4 (Ab183685, ABCAM), anti-CD8 (4SM15, eBiosciences) and anti-FOXP3 (12653, Cell Signaling Technologies) by IHC using the Ventana Discovery XT (Ventana; Tucson, Ariz.). Immunohistochemical staining was performed on the Discovery XT Automated IHC staining system using the Ventana DAB Map detection kit. Slides were manually counterstained with Hematoxylin (2 minutes), dehydrated and coverslipped. Images were acquired on the Aperio AT 2 slide scanner (Leica Biosystems; Buffalo Grove, Ill.) and analyzed using Indica HALO software (Indica Labs; Corrales, N. Mex.). H&E staining were performed by Histoserv, Inc (Germantown, Md., USA).

Both small and large tumors are infiltrated with CD4+ and CD8+ T cells at baseline without treatment. Tumors were then examined after treating with PSMAxCD3 or CD3-binding control. PSMAxCD3 treatment promoted the increase of CD8+ T cell frequency in both 50 mm³ and 200 mm³ tumors. In contrast, there was no significant effect on the frequency of CD4+ T cells. Additionally, FOXP3+ immunosuppressive Treg cell frequencies were similar across all groups (data not shown). Since T cells were present in both small and large tumors, the activity of these T cells after dosing with PSMAxCD3 or CD3-binding control was ascertained.

Flow cytometry analysis determined that CD8+ and CD4+ T cells in both small and large tumors upregulated cytolytic marker Granzyme B and proliferation marker Ki67 after PSMAxCD3 treatment (data not shown). Additionally, serum cytokine concentrations of IFN-γ, IL-2, and TNF-α were examined after PSMAxCD3 administration to indicate T cell activation. Tumor bearing HuT mice treated with PSMAxCD3 induced systemic cytokine production at 4 hours however, cytokine release returned to baseline concentrations by 72 hours, indicating a strong but transient T cell response (data not shown). In contrast, PSMAxCD3 in combination with anti-4-1BB resulted in enhanced cytokine release at 96 hours after treatment suggesting a durable T cell response. These results suggest that while the initial response may be sufficient to eliminate smaller tumors, which were already reduced in size by 48 hours, T cells cannot overcome the rapidly growing large tumor. Therefore, additional co-stimulation to boost the proliferation and expansion of tumor specific T cells might be required for an anti-tumor response in large tumors.

PSMAxCD3 with 4-1BB Co-Stimulation is Highly Efficacious Against Larger Tumors

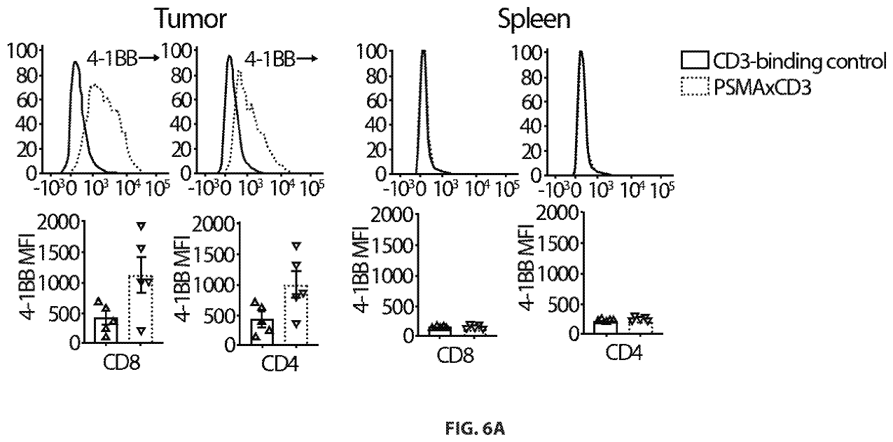

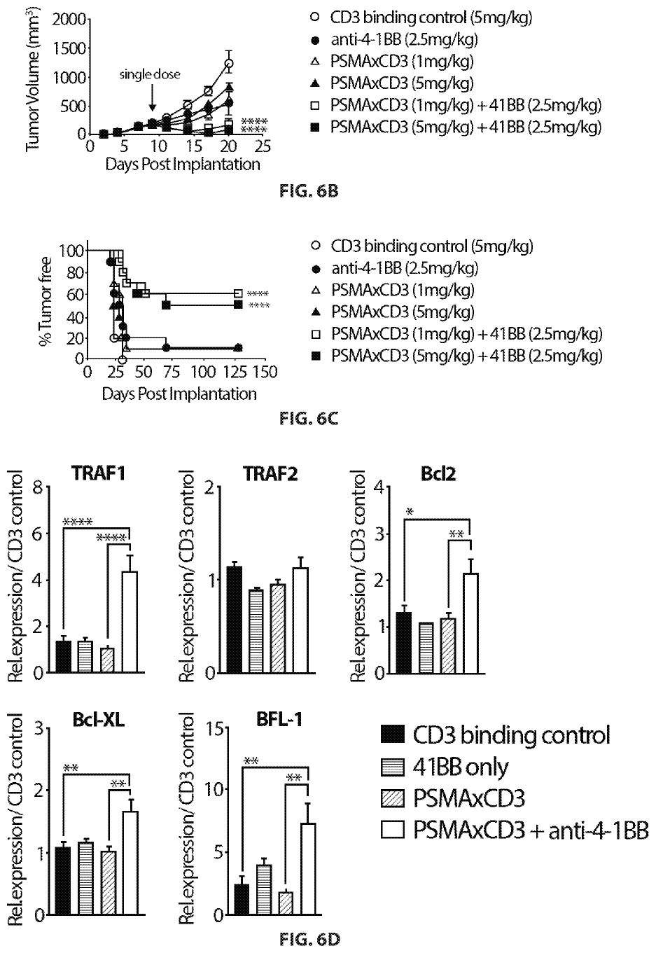

T cells from the larger tumor were examined for 4-1BB expression. Flow cytometry analysis demonstrated that PSMAxCD3 induces activation dependent 4-1BB surface expression that is restricted to intratumoral T cells, as expression is not observed on splenic T cells (FIG. 6A). Next it was determined whether co-stimulation of the 4-1BB pathway can increase anti-tumor efficacy in mice with a higher tumor burden. While PSMAxCD3 or anti-4-1BB alone showed some delay in tumor growth, mice treated with a single dose of PSMAxCD3 in combination with anti-4-1BB resulted in striking anti-tumor efficacy (FIG. 6B) and complete clearance of 50-60% of the tumors by day 60 (FIG. 6C). Notably, mice that received PSMAxCD3 in combination with anti-4-1BB did experience transient weight loss when given the higher dose of PSMAxCD3 in combination with anti-4-1BB. This transient weight loss can be mitigated by decreasing the dose of PSMAxCD3 from 5 mg/kg to 1 mg/kg in combination with anti-4-1BB without affecting overall anti-tumor efficacy (data not shown). Furthermore, mice treated with PSMAxCD3 in combination with anti-4-1BB showed elevated transcript expression of TRAF1 adaptor protein, which is essential to 4-1BB-induced activation pathways as well as upregulation of survival genes Bcl2, Bcl-XL (Bcl2l1), and BFL-1 (Bcl2a1a) (FIG. 6D).

PSMAxCD3 with 4-1BB Co-Stimulation Increases Expansion and Prolongs Survival of CD8 T Cells

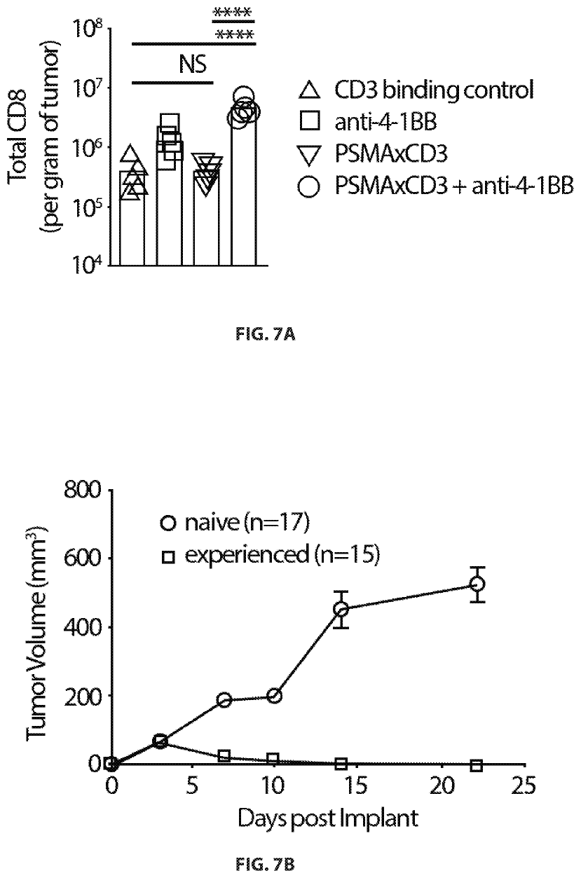

Serum cytokine release as an indication of T cell activation was assessed and mice treated with PSMAxCD3 in combination with anti-4-1BB showed enhanced and sustained cytokine induction even at 96 hours after treatment while cytokine concentrations had returned to baseline levels in mice treated with PSMAxCD3 alone (data not shown). Since survival genes are upregulated through the 4-1BB pathways, tumor infiltrating CD8 and CD4 T cells 96 hours after treatment were examined. Indeed, a prominent expansion of the CD8 T cell compartment was observed in mice treated with the combination treatment compared to CD3-binding control, anti-4-1BB or PSMAxCD3 alone (FIG. 7A). Furthermore, PSMAxCD3 in combination with anti-4-1BB enhanced proportions and total counts of Granzyme B+(data not shown) and Ki67+(data not shown) CD8 T cells, suggesting that combination treatment induces the expansion of tumor infiltrating T cells capable of cytotoxic activity and continued proliferation. Although total numbers of Treg cells were similar across treatment groups, the CD8 to Treg ratio was significantly enhanced due to the expansion of CD8+ T cells of mice that received the combination treatment (data not shown). Mice that cleared the large tumors with TRAMP-C2-hPSMA cells on the opposite flank were rechallenged. Compared to naive mice, the mice that received combination treatment were able to control secondary tumor challenge indicating the generation of tumor specific immunological memory (FIG. 7B and Table 2).

TABLE 2
Tumor Specific Immunological Memory

| | Tumor free |
|---|---|
| Treatment group from initial study | mice/ total |
| Naïve control mice not previously in study | 1/17 |
| Mice previously treated with PSMAxCD3 + | 15/15 |
| anti-4-1BB | |

Overall, the data demonstrated that while PSMAxCD3 is able to induce activation, cytokine production and proliferation of T cells in the short term, the combination with anti-mouse 4-1BB can prolong and enhance these effects to achieve anti-tumor efficacy even in established tumors. Furthermore, mice treated with PSMAxCD3 or PSMAxCD3+ anti-4-1BB were protected from secondary tumor challenge.

### CONCLUSIONS

A CD3-bispecific antibody targeting tumor antigen PSMA (PSMAxCD3) shows preclinical efficacy in multiple mouse models. PSMAxCD3 combined with anti-4-1BB achieves durable anti-tumor activity resulting in long term survival of mice, demonstrating that co-stimulation can enhance the potency of CD3-bispecific antibodies against advanced solid tumors.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A method of treating a cancer or inhibiting the growth of a tumor comprising administering to a subject in need thereof a therapeutically effective amount of each of (a) an anti-CD3/anti-PSMA bispecific antigen-binding molecule; and (b) an anti-4-1BB agonist.

2. The method of claim 1, wherein the cancer is selected from the group consisting of prostate cancer, kidney cancer, bladder cancer, colorectal cancer, and gastric cancer.

3. The method of claim 2, wherein the cancer is prostate cancer.

4. The method of claim 3, wherein the prostate cancer is castrate-resistant prostate cancer.

5. The method of claim 1, wherein the anti-CD3/anti-PSMA bispecific antibody and the anti-4-1BB agonist are administered separately.

6. The method of claim 1, wherein the anti-CD3/anti-PSMA bispecific antibody and the anti-4-1BB agonist are co-administered.

7. The method of claim 1, wherein the anti-CD3/anti-PSMA bispecific antibody is administered prior to, concurrent with or after the anti-4-1BB agonist.

8. The method of claim 7, wherein the anti-CD3/anti-PSMA bispecific antibody is administered prior to the anti-4-1BB agonist.

9. The method of claim 7, wherein the anti-CD3/anti-PSMA bispecific antibody is administered the same day as the anti-4-1BB agonist.

10. The method of claim 1, wherein the anti-CD3/anti-PSMA bispecific antibody is administered in combination with the anti-4-1BB agonist.

11. The method of claim 1, wherein the anti-4-1BB agonist is selected from a small molecule or an antibody.

12. The method of claim 11, wherein the anti-4-1BB agonist is an antibody selected from the group consisting of urelumab and utomilumab.

13. The method of claim 1, wherein the bispecific antigen-binding molecule comprises a first antigen-binding domain, and the first antigen-binding domain specifically binds human CD3 and comprises a heavy chain variable region (HCVR-1) amino acid sequence of SEQ ID NO: 2.

14. The method of claim 1, wherein the bispecific antigen-binding molecule comprises a second antigen-binding domain, and the second antigen-binding domain specifically binds human PSMA and comprises a heavy chain variable region (HCVR-2) amino acid sequence of SEQ ID NO: 1.

15. The method of claim 1, wherein the bispecific antigen-binding molecule comprises a first antigen-binding domain that specifically binds CD3 and comprises a HCVR-1 amino acid sequence of SEQ ID NO: 2 and a second antigen-binding domain that specifically binds PSMA and comprises a HCVR-2 amino acid sequence of SEQ ID NO: 1.

16. The method of claim 1, wherein the bispecific antigen-binding molecule comprises a common LCVR of SEQ ID NO: 3.

17. The method of claim 1, wherein the tumor volume is decreased relative to treatment in the absence of an anti-4-1BB agonist.

18. The method of claim 1, wherein tumor free survival is increased relative to treatment in the absence of an anti-4-1BB agonist.

19. The method of claim 1, wherein expression of TRAF1 in the tumor of a subject is increased by at least about 4 fold relative to TRAF1 expression in the tumor of a subject administered the anti-CD3/anti-PSMA bispecific antigen-binding molecule in the absence of an anti-4-1BB agonist.

20. The method of claim 1, wherein expression of Bcl2 in the tumor of a subject is increased by at least about 2 fold relative to Bcl2 expression in the tumor of a subject administered the anti-CD3/anti-PSMA bispecific antigen-binding molecule in the absence of an anti-4-1BB agonist.

21. The method of claim 1, wherein expression of BFL-1 in the tumor of a subject is increased by at least about 3 fold relative to BFL-1 expression in the tumor of a subject administered the anti-CD3/anti-PSMA bispecific antigen-binding molecule in the absence of an anti-4-1BB agonist.

22. The method of claim 1, wherein expansion of CD8+ T cells in the tumor of a subject is increased and/or survival of CD8+ T cells increases relative to CD8+ T cells in the tumor of a subject administered the anti-CD3/anti-PSMA bispecific antigen-binding molecule in the absence of an anti-4-1BB agonist.

23. A method of increasing expansion of CD8+ T cells in tumor tissue, the method comprising administering to a subject in need thereof a therapeutically effective amount of each of (a) an anti-CD3/anti-PSMA bispecific antigen-binding molecule; and (b) an anti-4-1BB agonist.

24. The method of claim 23, wherein the CD8+ T cells to Treg ratio increases in the tumor tissue of a subject treated with an anti-CD3/anti-PSMA bispecific antigen-binding molecule plus an anti-4-1BB agonist as compared to the CD8+ T cells to Treg ratio in the tumor tissue of a subject treated with an anti-CD3/anti-PSMA bispecific antigen-binding molecule in the absence of an anti-4-1BB agonist.

25. The method of claim 1, wherein subsequent exposure to tumor cells elicits a memory response in the subject treated with the anti-CD3/anti-PSMA bispecific antigen-binding molecule in the presence of an anti-4-1BB agonist.

26. A method of eliciting and/or enhancing a T cell response to a tumor, the method comprising administering to a subject in need thereof a therapeutically effective amount of each of (a) an anti-CD3/anti-PSMA bispecific antigen-binding molecule; and (b) an anti-4-1BB agonist.

27. A pharmaceutical composition comprising:
(a) a bispecific antigen-binding molecule comprising: (i) first antigen-binding domain that specifically binds human CD3 and comprises a HCVR-1 amino acid sequence of SEQ ID NO: 2 and (ii) a second antigen-binding domain that specifically binds human PSMA and comprises a HCVR-2 amino acid sequence of SEQ ID NO: 1;
(b) an anti-4-1BB agonist; and
(c) a pharmaceutically acceptable carrier or diluent.

28. The pharmaceutical composition of claim 27, wherein the bispecific antigen-binding molecule of part (a) comprises a common LCVR amino acid of SEQ ID NO: 3.

29. A radiolabeled bispecific antibody conjugate comprising a bispecific antigen-binding molecule that binds PSMA and CD3, a chelating moiety, and a positron emitter.

30. The conjugate of claim 29, wherein the bispecific antigen-binding molecule is covalently bonded to the chelating moiety, L, of formula (A):
-L-M_{z} (A)
wherein M is the positron emitter; and z, independently at each occurrence, is 0 or 1; and wherein at least one of z is 1.

31. The conjugate of claim 29, wherein the chelating moiety comprises desferrioxamine.

32. The conjugate of claim 29, wherein the positron emitter is ⁸⁹Zr.

33. The conjugate of claim 30, wherein -L-M is 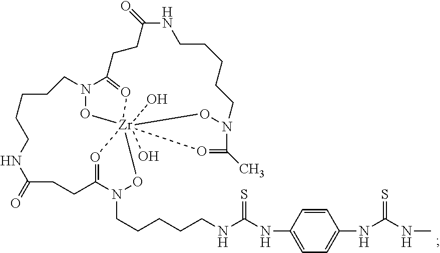
and wherein the positron emitter Zr is ⁸⁹Zr.

34. The conjugate of claim 30, wherein the bispecific antigen-binding molecule is covalently bonded to one, two, or three moieties of Formula (A).

35. The conjugate of claim 29, wherein the bispecific antigen-binding molecule comprises a first antigen-binding domain that specifically binds CD3 and comprises a HCVR-1 amino acid sequence of SEQ ID NO: 2 and a second antigen-binding domain that specifically binds PSMA and comprises a HCVR-2 amino acid sequence of SEQ ID NO: 1.

36. A method of imaging a tissue that expresses PSMA comprising administering a radiolabeled bispecific antibody conjugate of claim 29 to the tissue; and visualizing PSMA expression by positron emission tomography (PET) imaging.
